(19) United States
(12) Patent Application Publication
(10) Pub. No.: US 2005/0233403 A1
(43) Pub. Date: Oct. 20, 2005

(54) Human adipocyte cell populations and methods for identifying modulators of same

(75) Inventors: Michael John Stevenson, Boise, ID (US); James L. Kirkland, Brookline, MA (US)

Correspondence Address: LAHIVE & COCKFIELD, LLP., 28 STATE STREET, BOSTON, MA, 02109, US

(73) Assignee: AdipoGenix, Inc., Boston, MA (US)

(21) Appl. No.: 10/513,066

(22) PCT Filed: May 1, 2003
(86) PCT No.: PCT/US03/13758

§ 371 Date: May 31, 2005

Related U.S. Application Data
(60) Provisional application No. 60/377,500, filed on May 1, 2002.

(51) Int. Cl. C12Q 1/44

(52) U.S. Cl. 435/19

(57) ABSTRACT

The invention features methods of obtaining high-yield, essentially pure human predipocyte cultures. Cultures obtained according to the instant methodology are also featured as are methods of identifying adipogenic modulatory agents, e.g., high-throughput screening assays.

### RELATED APPLICATIONS

This application claims the benefit of prior-filed U.S. provisional patent application Ser. No. 60/377,500, entitled “Human Adipocyte Cell Populations and Methods for Identifying Modulators”, filed May 1, 2002 (pending).

### GOVERNMENT RIGHTS

This invention was made at least in part with government support under grant no. 1-R43-DK-54588-1 awarded by the National Institutes of Health. The government has certain rights in this invention.

### BACKGROUND OF THE INVENTION

Obesity is a well-established risk factor for many common diseases, including diabetes, coronary heart disease, hypertension, osteoarthritis, gallbladder disease, and colon, endometrial, and breast cancer. Visceral obesity is a particularly important risk factor for these diseases. Over one in three Americans are overweight, with a substantial economic effect. Annual direct healthcare costs of disease attributable to obesity in the U.S. were estimated at approximately 52 billion dollars in 1995, or 5.7% of our national health expenditure that year. Most of the direct costs (63%) were from type 2 diabetes. The potential market for anti-obesity treatments exceeds $33 billion in North America annually.

There is no cure for obesity. Current methods for managing obesity include appetite suppressant drugs, drugs that block intestinal absorption of nutrients, diets, surgery, and behavioral approaches. Results of these treatments have been disappointing: only a small percentage of weight is lost and this is typically regained. Existing drug and dietary treatments for obesity are only modestly effective. Current treatments for obesity focus on dieting, surgery, and drugs. There are problems associated with each of these approaches. People who diet are initially successful in losing weight. However, those who complete weight-loss programs lose approximately 10% of their body weight, only to regain two-thirds of it back within 1 year and almost all of it back within 5 years. Strict dieting alone results in loss of lean tissue as well as fat. When obese experimental animals are starved to death, some preservation of fat, despite utilization of brain and heart for energy, is observed. Surgery involves many complications and is often only used in the morbidly obese as a last resort.

Drugs that are being developed can be broken down into three categories: drugs that target the brain, drugs that affect fat absorption by the intestines, and drugs that affect fat cells directly. Drugs that target the brain rarely affect only one CNS pathway. There are many potential CNS side-effects, e.g., on memory, behavior, and sexual function in addition to systemic effects (e.g., cardiac and pulmonary dysfunction). CNS appetite suppressant drugs share some of the drawbacks of dietary approaches including loss of lean as well as fat mass and lack of effect on specific fat depots. Drugs that affect fat absorption also tend to affect absorption of other critical nutrients and often cause diarrhea or other gastrointestinal side-effects. These drugs also share some of the drawbacks of dietary approaches. Drugs that affect fat cells directly (e.g., β3 agonists) have sometimes been developed initially using animal models and later have proven to be less effective in humans without substantial expenditures for further development.

The group of drugs most likely to be effective in treating all forms of obesity and with the best side effect profiles are likely to be drugs that act directly on fat tissue to modify fat cell size or number. Currently, strategies to discover drugs acting at the level of the fat cell usually involve screening assays in animal models, animal aneuploid cell lines, or freshly-derived tissue from human subjects. Several problems are associated with these approaches including: (1) Fat cells from humans are very different from rodent fat cells or preadipocyte-like aneuploid cell lines. Consequently, drugs that appear promising in such models have proven ineffective in human fat tissue. (2) Preadipocyte-like aneuploid cell lines exhibit a number of differences from euploid cells. For example, 3T3-L1 cells contain anywhere from a few to over 200 chromosomes, are immortal in culture, differentiate under conditions in which rat or human primary culture preadipocytes do not, and exhibit differences from primary cultured preadipocytes in the appearance of catecholamine responsiveness, capacity for de novo triglyceride synthesis, and hormone-sensitive lipase activity during differentiation. For these reasons, it has been stated that findings in cell lines need to be verified in euploid preadipocytes (MacDougald, O. A. et al. (1995) Annu. Rev. Biochem 64:345-73; Smas, C. M. et al. (1995) Biochem J. 309, 697-710; Cornelius, P. et al. (1994) Annu. Rev. Nutr. 14:99-129) (3) Use of human fat cells for developing new drugs presents problems: sufficient human fat tissue for assays is difficult to obtain, particularly in quantities sufficient for high throughput assays, and fat tissue deteriorates rapidly.

Human preadipocytes are extremely valuable as a drug development system for the reasons given above, however, human preadipocytes have not been used extensively to date due to a host of problems including (1) inability to obtain adequate amounts of fat tissue from which to isolate preadipocytes (2) difficulty in inducing adipocyte differentiation (even under optimized differentiation culture conditions (Hauner, H., et al. (1989) J. Clin. Invest. 84:1663-70; Van de Venter, M., et al. (1 994) J. Cell. Biochem. 54:1-10), (3) inability to subculture human preadipocytes that retain the capacity to differentiate (e.g., as compared to in primary cultures). Thus there exists an art-recognized need for improved methods of obtaining human preadipocyte cells, in particular, for obtaining high yields of substantially pure preadipocyte cultures that maintain a high differentiative capacity.

### SUMMARY OF THE INVENTION

The present invention features improved processes for isolating and culturing human preadipocytes. In particular, the invention features isolation processes for obtaining high yield, substantially pure human preadipocyte cultures and/or subcultures. Also featured are optimized techniques for differentiating human adipocytes. Human preadipocytes can thus be prepared in an inexpensive, consistent, and effective process yielding differentiated human preadipocytes in quantity. Human preadipocytes prepared according to the methodology of the instant invention are particularly amenable for use in high-throughput drug screening efforts.

### DETAILED DESCRIPTION OF THE INVENTION

The instant invention features high-yield, essentially pure, human preadipocyte and adipocyte cell populations and methods for obtaining said cell populations. In particular, the invention features methods for obtaining high yields of preadipocytes from a relatively limited source of human tissue, methods of culturing said preadipocytes such that essentially pure cultures are obtained, and optimized methods of differentiating said pure populations such that highly differentiated adipocyte cultures are obtained. The preadipocyte and adipocyte cultures of the invention are particularly suited for use in high throughput screening assays for identifying modulators of fat cell replication, differentiation and function. Preferred methods of the instant invention feature the clonal expansion and subsequent differentiation of human preadipocytes (or high yield preadipocyte populations) such that reproducible high throughput screening assays can be carried out.

In one aspect, the invention features high-yield, essentially pure, human, preadipocyte populations. In another embodiment, the invention features highly-differentiated, human, adipocyte populations. In yet another embodiment, the invention features methods of identifying modulators (e.g., inhibitors) of human preadipocyte/adipocyte differentiation using the above-mentioned cell populations. Preferred methods of identifying modulators (e.g., inhibitors) of human preadipocyte/adipocyte differentiation include assaying cell populations for a detectable fluctuation in fatty acid uptake (e.g., assaying cell populations for a detectable inhibition of fatty acid uptake). Modulators identified according to -the methodology of the instant invention are also featured. Preferred modulators of the invention are those that act directly on fat cells and/or fat tissue. Additional preferred modulators are those that act in a depot-specific manner.

Prior to describing the invention, it may be helpful to an understanding thereof to set forth definitions of certain terms to be used hereinafter.

The term “preadipocyte” refers to a cell existing in or isolated from fat tissue which is capable of replicating yet is committed to the adipogenic phenotype (i.e., is committed to differentiate into an adipocyte or fat cell). In their undifferentiated state, cultured preadipocytes resemble fibroblasts (i.e., have a fibroblast-like morphology). In particular, they exhibit a flattened, adherent morphology and contain very little microscopically-detectable lipid. The term “human preadipocyte” refers to a preadipocyte existing in or isolated from human fat tissue.

The term “adipocyte” refers to a cell existing in or derived from fat tissue which is terminally differentiated. In their differentiated state, adipocytes assume a rounded morphology associated with cytoskeletal changes and loss of mobility. They further accumulate lipid as multiple small vesicles that later coalesce into a single, large lipid droplet displacing the nucleus. The term “human adipocyte” refers to an adipocyte existing in or isolated from human fat tissue.

The term “partially-differentiated preadipocyte” refers to a preadipocyte which exhibits one or more markers of differentiation, for example, accumulation of cytoplasmic lipid, but is still capable of replication.

The phrase “essentially pure” refers to a cell population, e.g., a human preadipocyte population, that has been isolated from its natural source (e.g., has been isolated or purified from fat tissue, for example, from human fat tissue) and, through a purification step or series of purification steps, has been separated from other cells (e.g., non-preadipocyte cells) and cellular debris. An essentially pure cell population, as defined according to the instant invention, is at least 90% pure, i.e., at least 90% of the cells are of the desired cell type (e.g., human preadipocytes) and less then 10% are contaminating (e.g. non-preadipocyte) cells. In a preferred embodiment, an essentially pure cell population (e.g., an essentially pure preadipocyte population) is at least 95% pure. In a more preferred embodiment, an essentially pure cell population (e.g., an essentially pure preadipocyte population) is at least 96%, 97%, 98%, 99% or 100% pure). Purity of a preadipocyte culture is most easily determined after the culture has been differentiated into adipocytes (i.e., by culturing for appropriate times in the presence of differentiation-inducing and differentiation promoting agents, as defined herein). Any marker of differentiation, as defined herein, can be used to determine the percentage of cells that have differentiated. For example, where 100% of the cells in a culture of the invention have accumulated lipid droplets to an appreciable size (i.e. 100% of the cells are adipocytes), it can be concluded that 100% of the cells were preadipocytes of the original culture were preadipocytes, i.e., the starting culture was 100% preadipocytes or 100% pure.

The phrase “capable of replication” is used to refer to cells (e.g., preadipocytes or partially-differentiated preadipocytes) capable of undergoing cell division. Cell division can be determined or assessed histologically, by flow cytometry, indirectly (e.g., as an increase in DNA synthesis, for example, an increase in radiolabeled thymidine into a cell or cell population).

The term “passage” refers to the transfer or transplantation of cell, with or without dilution, from one culture vessel to another. It is understood that any time cells are transferred from one vessel to another, a certain portion of the cells may be lost and, therefore, dilution of cells, whether deliberate or not, may occur. The term passage is synonymous with the term subculture. The number of passages a population can proceed through is characteristic of the quality of the preparation.

The phrase “high-yield” means a yield of 10⁶ cells from 25 g or less of isolated human fat, preferably a yield of 10⁶ cells from 10 g or less of isolated human fat, more preferably a yield of 10⁶ cells from 5 g or less of isolated human fat, and most preferably a yield of 10⁶ cells from 1 g or less of isolated human fat. The phrase “high yield” refers to the result of processes of the instant invention as compared to published “low yield” processes, for example, processes yielding 10⁶ cells from approximately 70 g of isolated human fat.

The phrase “enhanced differentiative capacity” means at least a 2-fold, preferably a 3, 4 or 5-fold, more preferably a 6-, 7-, 8-, 9-, 10-fold or greater improvement in extent of human preadipocyte differentiation beyond established, published methods.

The phrase “differentiation-inducing agent” refers to a compound or agent that initiates or stimulates the differentiation of preadipocytes into adipocytes. Preferred “differentiation-inducing agents” include but are not limited to insulin, insulin-sensitizing agents, substrates for lipid synthesis, PPAR ligands (e.g., natural ligands, for example, prostaglandin J₂, and synthetic ligands, for example, thiazolidinediones, and the like). The phrase “differentiation-promoting agent” refers to a compound or agent that enhances or accelerate the differentiation of preadipocytes into adipocytes. Preferred “differentiation-inducing agents” include but are not limited to insulin, insulin-sensitizing agents, substrates for lipid synthesis, PPAR ligands, and the like.

“Differentiation-inducing agents” or “differentiation-promoting agents” vary considerably in effectiveness but share common effects on several cellular signaling pathways including, but not limited to: (1) tyrosine kinase pathways (e.g., IGF-1-mediated tyrosine kinase pathway); (2) adenylyl cyclase/phosphodiesterase signaling pathways; (3) steroid/thyroid/peroxisome proliferator activated (PPAR)/retinoid nuclear receptors signaling pathways; and (4) protein kinase signaling pathways (MacDougald, O. A. et al. (1995) Annu. Rev. Biochem 64:345-73; Smas, C. M. et al. (1995) Biochem J. 309, 697-710; Cornelius, P. et al. (1994) Annu. Rev. Nutr. 14:99-129).

Following induction of differentiation through these signal transduction pathways, coordinated changes in the expression of over 600 genes occurs leading to the acquisition and maintenance of the fat cell phenotype (MacDougald, O. A. et al. (1995) Annu. Rev. Biochem 64:345-73; Smas, C. M. et al. (1995) Biochem J. 309, 697-710; Cornelius, P. et al. (1994) Annu. Rev. Nutr. 14:99-129). These changes in differentiation-dependent gene expression are orchestrated by several transcription factors including CCAAT enhancer binding proteins (C/EBPα, β, and γ), PPARγ, and others (reviewed in MacDougald, O. A. et al. (1995) Annu. Rev. Biochem 64:345-73; Smas, C. M. et al. (1995) Biochem J. 309, 697-710; Kirkland, J. L., et al.(1997) J. Amer. Geriatr. Soc. 45:959-67). Overexpression of some of these transcription factors, including C/EBPα and PPARγ, is sufficient to induce the differentiation of preadipocytes (Lin, F. T., et al (1994) Proc. Natl. Acad: Sci. USA 91:8757-8761; Hu, E. et al. (1995) Proc. Natl. Acad. Sci. USA 92:8956-60; Wu, Z., et al. (1995) Genes Defer 9:2350-63; Yeh, W. C., et al. (1995) Genes Devel. 9:168-81).

Markers of differentiation include (presented in order of detectable changes in expression): (1) cytoskeletal genes; (2) lipoprotein lipase (LPL) and collagen isoforms; (3) adipocyte fatty acyl binding protein (aP2) and glycerol-3-phosphate dehydrogenase (G3PD); and/or (4) the insulin sensitive glucose transporter (GLUT4), angiotensinogen (ang), apolipoprotein E (apoE), leptin, adipsin (complement factor D), protein C3, factor B, and other genes occur that contribute to the endocrine/paracrine function of adipose tissue. Increased fat cell size, which is dependent on the balance between rates of lipogenesis (and differentiation) and lipolysis, is also a detectable marker of adipocyte or fat cell differentiation.

The term “fat depot” refers to a deposit of fat cells existing within animal, preferably human, tissue comprising essentially adipocytes and/or preadipocytes. Fat depots can be peripheral or visceral. Preferred fat depots include, but are not limited to subcutaneous fat depots, omental fat depots and mesenteric fat depots. The phrase “depot-specific effect” or “fat depot-specific effect” refers to an effect, for example a biological or physiological effect, that is particular to one or more fat depots (or cells obtained from said depot or depots) but not common to fat cells or fat depots of all origins.

The term “mesenteric” means of or having to do with the mesentery. The mesentery is any membranous fold attaching various organs to the body wall. Specifically, mesentery refers to the peritoneal fold attaching the small intestine to the dorsal abdominal wall. For the purposes of the instant invention the term mesenteric is used to describe fat that develops around the mesentery.

The term “omental” pertains to an area around the omentum. The omentum is a fold of peritoneum extending from the stomach to adjacent organs of the abdominal cavity. For the purposes of the instant invention the term omental is used to describe omental fat, or omental fat depots, which describes fat that develops around the omentum.

The term “subcutaneous” is an art recognized term that refers to the location below the skin. In terms of this invention, the term refers to subcutaneous fat, i.e., fat located in depots below the skin.

As used herein, a fat cell, preadipocyte or adipocyte “modulator” or “modulatory” compound or agent is a compound or agent that modulates at least one biological marker or biological activity characteristic of fat cells and/or fat tissue. In preferred embodiments, compounds or agents of the invention modulate at least one of (1) differentiation-specific gene expression, (2) lipid metabolism (e.g., lipogenesis and/or lipolysis), (3) fatty acid uptake, and/or (4) accumulation of cytoplasmic lipid.

The term “serum-free” refers to a medium free of animal serum, free of partially-purified or characterized animal serums, serum substitutes and the like. Preferably, a serum-free medium is free of bovine serum, fetal bovine serum, etc.

Where ranges are recited herein, all intermediate values within the ranges are expressly within the scope of the invention.

Various aspects of the invention are described in further detail in the following subsections:

I. Isolating and Subculturing of Human Preadipocytes:

The instant invention features methods of isolating and culturing or subculturing human preadipocytes that are significantly improved over methods previously published. In particular, the methods of the instant invention feature isolation of essentially pure, high-yield, human preadipocyte cell populations that are capable of undergoing multiple rounds of division while maintaining differentiative capacity. Preadipocyte cultures of the instant invention maintain differentiative capacity for at least 4 passages and preadipocytes capable of differentiating after as many as 25 passages have now been obtained. This is quite significant when compared to published reports of human preadipocytes losing differentiative capacity after 3-4 passages. The significant increase in replicative capacity of differentiation-competent cells is due to a combination of the purity of preadipocyte cultures at outset (i.e., no “overgrowth” of contaminating cell types) and the improved differentiation culture methodology of the instant invention.

This ability to amplify preadipocytes that maintain differentiative capacity results is key to being able to perform screening assays for modulators of adipocyte function. For example, even a 10-fold amplification (i.e., 10 passages) of human preadipocytes allows expansion of a single flask of cells into 1024 flasks. Adipocytes isolated and subcultured as described herein can further be cryopreserved (“banked”) and passaged again after thawing. A summary of the preferred isolation methodology of the instant invention is as follows:
A. Cells are isolated from tissue biopsies obtained during surgery (e.g., abdominal surgery). Alternatively tissue, such as for example omental fat tissue, may be obtained by laparoscopy. Preferably, the time from tissue harvest to isolation is kept to a minimum. In one embodiment, the time from tissue harvest to isolation is no greater than 24 hours. In another embodiment, the time from tissue harvest to isolation is no greater than 20 hours. In another embodiment, the time from tissue harvest to isolation is no greater than 16 hours. In another embodiment, the time from tissue harvest to isolation is no greater than 12, 8, 4 or 2 hours.
B. The tissue is transported from the operating room in a buffered transport medium (e.g., a PBS buffered medium or HBSS/sodium bicarbonate buffered medium) containing antibiotics (e.g., gentamicin, amphotericin, penicillin, and/or streptomycin) and L-glutamine.
C. The tissue is then sectioned into pieces between 5-10 mg and placed in centrifuge tubes (e.g., 50 ml centrifuge tubes) containing digestion medium. A preferred digestion medium is a buffered medium (e.g., PBS or HBSS buffered medium) including collagenase, BSA (e.g., fatty-acid free BSA), and, optionally, L-glutamine. Preferably 3 mg of collagenase is used per gram of tissue.
D. Omental tissue is processed first, since digestion of omental tissue takes longer than mesenteric or subcutaneous tissue. Subcutaneous tissue is processed next, followed by mesenteric. The tissue is finely minced (pieces approximately 1-3 mm in size) using sharp sterile scissors, then vortexed thoroughly and placed in a shaking water bath at 37° C. (water level high enough to submerge all of tissue in tubes).
E. The tubes are thoroughly vortexed every 10 minutes until the tissue is fully digested.
F. The solution is then filtered through a gauze filter (Steri-Pad 4×4 inch, Johnson and Johnson, NJ) and spun at 1000×g for 10 minutes.
G. The resulting pellet is resuspended in an erythrocyte-lysing buffer containing potassium bicarbonate, EDTA, and ammonium chloride.
H. The tubes are placed in a shaking water bath at 37° C. for five minutes and then spun at 1000×g for 10 minutes.
I. The pellet is then plated overnight in plating medium, which consists of DMEM/F12, HEPES, sodium bicarbonate, penicillin, streptomycin, L-glutamine, and NuSerum (a semi-artificial serum supplement available from BD BioSystems, Inc., Bedford, Mass.). (Alternatively, cells (e.g., cells obtained from laparoscopy) can be plated in α-MEM-based plating medium as follows. Cells are plated in a 100 mm cell culture dish for a minimum of 16 hours, and not more than 24 hours, at 37° C., 5% CO₂ in α-MEM 10 medium (alpha MEM sodium bicarbonate buffered medium supplemented with 10% fetal bovine serum and antibiotics, streptomycin and penicillin, see Table 7) prior to subsequent processing in artificial, substantially animal product free medium (such as for example, NuSerum available from BD BioSciences, cat. # 35-5504) in order to obtain optimum yield.)
J. After 6-24 hours of adherence, the plates are then washed and trypsinized. Preferably the cells are allowed to adhere for 16-24 hours prior to trypsinization, more preferably for about 18 hours prior to trypsinization.
K. The trypsinized cells are centrifuged at 1000×g for 10 minutes, and the resulting pellet is resuspended in DMEM/F12 plating medium (as described in Table 10). The cell density is determined using trypan blue and a hemocytometer, counting all four quadrants. # of cells =(cell count/4)×2×(1×10⁴)×(# ml cells are suspended in).
L. The cells are plated at a density of5.0×10⁴cells/cm², e g., in T-25 flasks. The cells are grown to confluence and either passaged 1:2 for further cell replication, frozen in liquid nitrogen storage, or further cultured in differentiation medium.
M. When passaging cells, preadipocytes will have a fibroblast-like appearance. During the isolation procedure, it is possible to have contaminating cells present, especially in omental preadipocyte preparations. These contaminating cells are flat and have a rounded morphology, and in many cases have a raised circular center, giving these cells a “fried egg” appearance. The percentage of contaminating cells can range from zero to as much as 50%, in rare cases. These cell types are removed during the passaging process when the preadipocyte population reaches greater than 75% confluency. The contaminating cells are removed by differential trypsin treatment. The dish or flask of adhered cells is washed once with PBS/EDTA (Table 8), then trypsin in PBS/EDTA (Table 9) is added for approximately one minute, with brief swirling every 20 seconds. The trypsin solution is then removed, and remaining attached cells are washed again in PBS/EDTA. The cells are then detached by addition of trypsin in PBS/EDTA, and are replated into a new dish or flask of similar size and allowed to grow. If contaminating cells are still present after a few days of cell growth, the procedure is repeated when the preadipocyte population is greater than 75% confluency.

II. Differentiation of Human Preadipocytes into Adipocytes:

The differentiation methods of the instant invention result in dramatically improved human adipocyte cultures. In particular, human adipocytes cultured according to the methods of the instant invention are uniform and highly differentiated. Moreover, the isolation and differentiation methods of the invention are suitable to isolation of fat from each of visceral, omental fat and mesenteric fat depots. Compared to published methods, cultures of omental adipocytes isolated and cultured according to the methods of the instant invention exhibit at least 4-fold enhanced differentiation (as determined according to the fatty acid uptake assay described herein) and cultures of visceral adipocytes exhibit at least 100-fold enhanced differentiation. A summary of the preferred differentiation methodology of the instant invention is as follows:
A. Preadipocytes are seeded in PM at 3×10⁴ cells/cm2 to insure 100% confluency upon adherence to the cell culture dish or plate.
B. 48 to 120 hours after seeding (optionally with a changing of PM every 2-4 days), plating medium is removed and cells are exposed to a serum-free differentiation medium (DM) consisting of the following basic components:

DMEM/F12, HEPES, sodium bicarbonate, penicillin, streptomycin, L-glutamine, biotin, human insulin, panthothenic acid, dexamethasone, and triiodo-L-thyronine. Exemplary concentrations are as follows:
DMEM:F12 (1:1),
15 mM HEPES,
15 mM NaHCO3,
2 mM glutamine,
   33 μM biotin,
   0.5 μM insulin,
   17 μM pantothenate,
   0.1 μM dexamethasone,
   2 nM triiodothyronine, and
   antibiotics.
   In other embodiments, cells are exposed to differentiation medium for two to ten days or two to fourteen days.
C. In addition, a number of other factors are preferably included in the DM at various concentrations, for example transferrin, fetuin, rosiglitazone, and isobutylmethylxanthine (IBMX). Exemplary concentrations are as follows:
   the thiazolidinedione rosiglitazone (up to 1 μM),
   fetuin (up to 1 g/L),
   transferin (up to 10 mg/L), and
   isobutylmethylxanthine (up to 600 μM).
D. The medium is changed every two to three days as preadipocytes differentiate into adipocytes. After cells have attained a rounded morphology, DM can be changed to DM without IBMX. After cells have become more rounded and/or triglyceride deposits become visible (i.e., microscopically visible), DM without IBMX can be changed to DM without IBMX, rosiglitazone, insulin or dexamethasone.

The isolation/differentiation method described in Examples 1 and 2 was compared to a generally-used, published method (Hauner, H., et al (1989) J. Clin. Invest. 84:1663-70; Hauner, H., et al. (1995) Eur. J. Clin. Invest. 25:90-96)). The published method (as most recently modified) involves collagenase digestion (with FBS), filtration, treatment with an erythrocyte lysis buffer, and plating in a medium that contains FBS. Confluent cells are then exposed to a differentiation medium that contains: DMEM:F12 (1:1), 15 mM HEPES, 15 mM NaHCO₃, 33 μM biotin, 0.5 μM insulin, 17 μM pantothenate, 0.1 μM dexamethasone, 0.2 nM triiodothyronine, and antibiotics. The methodology of the present invention differs in many respects from the published method. For example, different mincing procedures are used for different fat depots. BSA is used in the collagenase solution both to increase yield and because exposure to FBS inhibits subsequent differentiation of human preadipocytes. Different filtering material is used. As in the published method, digests are treated with an erythrocyte lysis buffer, since prolonged exposure to erythrocytes inhibits preadipocyte differentiation. Cells are plated in a medium containing a semi-artificial serum supplement instead of FBS to prevent effects of exposure to FBS on subsequent differentiation. Plating density is different. Cultureware was selected that enhances yield and differentiation. Unlike the published method, human preadipocytes are trypsinized and are replated after 18 hours. It has been determined that within about 18 hours after the initial plating, contaminating cells (e.g., macrophages mesothelial cells) strongly adhere but adipocytes only loosely adhere. Subsequent mild trypsination results in detachment of preadipocytes but not contaminating cells, increasing culture purity. The trypsination/replating step also facilitates accurate cell counting (i.e., non-cell contaminants, tissue debris, cellular debris (e.g., cell membranes) and the like are removed).

Cells are plated to insure confluence. Confluent cells are exposed to a serum-free differentiation medium that contains the same basic components as the medium used in published methods (although at different concentrations) as well as a number of other factors, including thiazolidinediones, fetuin, transferin, and isobutylmethyl-xanthine pretreatment, among others. Serum free-medium is used as it has been determined that the published serum-containing media inhibit the differentiation of human adipocytes.

Although some of these approaches have been used in rodent preadipocyte cell lines and, in some cases, human preadipocytes, it is the combination of protocol modifications that results in the instant effective and rapid method for differentiating human preadipocytes.

Unlike the published method, 3^{rd}, 4^{th}, or later passage cells are routinely used, thus greatly increasing yield. Sufficient differentiation of 1 0th passage cells is obtained that signal-to-noise ratio is satisfactory even in these cells. The published method is quite ineffective for cells that have been subcultured (Entenmann, G., et al. (1996) Am J. Physiol. 270:C1011-6)). Typically, primary culture cells need to be exposed to the published differentiation medium for over 18 days for cells to accumulate lipid droplets. In fourth subculture human preadipocytes, cells differentiated under the conditions described herein accumulate extensive lipid within 10 days. Parallel treatment of similarly subcultured cells with the published method resulted in very little adipogenesis.

Advantageously, isolated depot specific preadipocytes may be cryopreserved until thawed for use. For example, after isolation and growth as described above, preadipocytes are detached from the flask or dish using trypsin in PBS/EDTA. Cells are spun down and resuspended in ice-cold freezing medium (DMEM/F12, HEPES and bicarbonate buffered medium plus 10% glycerol), placed at −70° C. overnight and then transferred to liquid nitrogen for storage. For subsequent use, cells may be thawed in a water bath at 37° C., then slowly diluted in plating medium to lessen/prevent osmotic shock. Cells may be spun down to remove the glycerol and then resuspended in plating medium for culture, or alternatively allowed to adhere for 4 to 6 hours at 5% carbon dioxide/37° C., then changed with fresh plating medium for culture.

III. Screening Assays:

The human preadipocyte and/or adipocyte populations of the instant invention are particularly amenable to use in high-throughput drug screening efforts to identify compounds that act directly on fat cells (and/or their targets). In particular, the human preadipocyte and/or adipocyte populations of the instant invention are useful in assaying for agents that modulate the lipid content of adipocytes or fat cells (i.e., agents that modulate fatty acid uptake and/or lipid accumulation) and in assaying for agents that modulate adipocyte or fat cell differentiation. Preferably the human preadipocyte and/or adipocyte populations of the instant invention are used in assays to identify agents that inhibit the lipid content of adipocytes or fat cells (i.e., agents that inhibit fatty acid uptake and/or lipid accumulation) and/or in assays to identify agents that inhibit differentiation of adipocytes or fat cells. Compounds so identified are particularly useful for the development of drugs which directly act on fat tissue in vivo.

Accordingly, the present invention features rapid high-throughput screening assays or methods (“HTS assays”) for identifying compounds or agents that effect or modulate human preadipocyte and/or adipocyte fatty acid incorporation and/or differentiation. Sensitive and reproducible HTS assays featuring human preadipocyte and/or adipocytes are possible as a result of (1) the improved methods for isolating and purifying human preadipocytes; (2) inexpensive, consistent, and effective methods yielding differentiated human preadipocytes in quantity; and (3) methods developed for sensitive measurement of changes in fatty acid content. The improved isolation, culture and differentiation methodology of the instant inventions also make it possible to established a “bank” of stored (i.e., cryopreserved) human preadipocytes such that a steady and reproducible supply is available.

Hits (e.g., lead compounds) identified as described herein can further be subjected to a variety of secondary screening assays. In certain embodiments, secondary screening assays are utilized to validate a hit or lead as a fat modulatory compound (e.g. an effector of fat accumulation). In other embodiments, secondary screening assays are utilized to determine pathways affected by a hit (e.g., lead compound) and/or to identify a target (e.g., cellular or molecular target) of the hit or lead.

Targets in the pathway(s) resulting in adipogenesis, fatty acid uptake and/or triglyceride synthesis/storage (lipogenesis), or triglyceride breakdown (lipolysis) and/or fatty acid oxidation may be also be identified using the methods provided herein. Target identification allows rational design of agents and drugs useful for preventing or reversing obesity. Identification of an agent and its target allow identification of effector sites, facilitating design of more effective drugs.

Preferred features of the screening assays of the instant invention are consistency and sensitivity.

A. Test Compounds

Test compounds include small molecules, peptides, polypeptides and nucleic acid molecules or libraries of such molecules. The test compounds of the present invention can be obtained using any of the numerous approaches in combinatorial library methods known in the art, including: biological libraries; spatially addressable parallel solid phase or solution phase libraries; synthetic library methods requiring deconvolution; and synthetic library methods using affinity chromatography selection.

Libraries of compounds may be presented in solution, or on beads or chips or the like. Exemplary libraries include, but are not limited to synthetic chemical product libraries and natural product libraries (e.g., partially deconvoluted natural product libraries).

B. High Throughput Screening Assay Formats

B 1. Identification of Lead Compounds

The high-throughput screening assays (“HTS assays”) for the identification of compounds or agents that modulate fat cell activity, function or phenotype have been developed featuring either human preadipocytes, partially-differentiated adipocytes or adipocytes, isolated and cultured as described above. The HTS assays preferably feature identifying compounds that exhibit an effect on fatty acid uptake/accumulation according to the fatty acid-based celluomic assay described herein (see Example 4). Although cell-based screens are more technically demanding than target-based screens, this approach offers several advantages as a primary screen. Use of specific targets (i.e., molecular targets or pathways) obviously narrows the range of effective therapeutic classes likely to be discovered, while potentially novel compounds acting on unknown targets can be discovered using a cell-based approach. Moreover, no information is yet available about molecular targets likely to result in discovery of fat depot-specific anti-obesity agents in the human, and the time, effort, and risk involved in identifying cellular targets is considerable. Indeed, the cell-based approach may lead to identification of novel targets for use in later screening efforts. By allowing the human adipocyte to “select” the gene and protein target, the drug discovery process will be accelerated.

In certain assay formats, preadipocytes are contacted with a test compound in the presence of differentiation medium, cultured for a predetermined amount of time in the presence of the test compound and the differentiation medium (e.g., for an amount of time sufficient to allow differentiation into adipocytes) and assayed at the end of the predetermined time for fatty acid uptake/accumulation. The skilled artisan will appreciate, however, that differentiation of adipocytes is not essential to the methodologies of the instant invention. In certain embodiments, compounds that result in a lessening or reduction of fatty acid uptake/accumulation are selected as potential lead compounds or “hits”. Such compounds are useful, for example, as therapeutic agents (or in the development of therapeutic agents) for effecting weight loss, treating obesity, etc. In other embodiments, compounds that result in an increase of fatty acid uptake/accumulation are selected as potential lead compounds or “hits”. Such compounds are useful, for example, as therapeutic agents (or in the development of therapeutic agents) for the treatment of cachexia and/or anorexia. Additionally, hits or leads identified according to the methodologies of the present invention may be useful, for example, as therapeutic agents (or in the development of therapeutic agents) for the treatment of reduced insulin sensitivity, insulin resistance, diabetes (e.g., Type II diabetes), and the like.

In other assay formats, human adipocytes (in differentiation medium) are contacted with a test compound, cultured for a predetermined amount of time in the presence of the test compound (e.g., for an amount of time sufficient to allow loss of lipid from lipid droplets or de-differentiation of adipocytes) and assayed at the end of the predetermined time for fatty acid uptake/accumulation. Compounds that result in a lessening or reduction of fatty acid uptake/accumulation are selected as potential lead compounds or “hits”.

HTS assays are preferably carried out using 96-well or 384-well assay plates, although other configurations (e.g., other multi-well formats or coverslip formats) are within the scope of the instant invention.

In preferred embodiments, compounds or agents are screened for both toxicity (e.g., in vitro toxicity) and in vitro biological activity. Non-toxic compounds (e.g., those that are non-toxic at the at the low micromolar level) (potency (ED₅₀)) exhibiting in vitro biological activity (e.g., efficacy (maximum response)) are preferred. Toxicity screens can be performed utilizing preadipocytes or adipocytes isolated according to the present invention. Alternatively, compound toxicity can be screened using non-adipocyte cells, for example, using fibroblasts or hepatocytes. Toxicity screens using preadipocytes or adipocytes can be performed prior to biological activity assays, subsequent to biological activity assays, or in parallel with biological activity assays (i.e., in parallel cultures). In a preferred embodiment of the instant invention, toxicity screens using preadipocytes or adipocytes are performed in the same cultures as biological activity screens (see e.g., Example 6 and 7). Acceptable toxicity of the agent or modulator is less than 20% in the cell assay, more preferably the toxicity is less than 15%, most preferably the toxicity is less than 10%.

The two formats are designed to identify hits that potentially target different stages of adipogenesis. Hits detected after early addition may affect differentiation and/or fat accumulation, while those detected from late addition may affect lipogenesis or lipolysis. Therefore, different classes of drugs are likely to result from this screening strategy. Preferably, “hits” (or positive scoring library components) from this assay will have a response of ≦50% of the control value at 3μM synthetic compound or 0.05× dilution of natural product with minimal toxicity in the cell-based toxicity assay.

Preferably, hits are automatically flagged (plate number, row, and column) with the instrument control and data analysis software. Library components are preferably screened in triplicate, and are checked for fluorescent properties that could interfere with interpretation of assay results. Potential false positives from this screen could arise through a variety of mechanisms. For example, compounds that interact with the fluorescent fatty acid itself and block its uptake could give a positive response in this assay. Also, compounds that are sequestered in fat droplets with the fluorescent fatty acids and that quench their fluorescence could result in false positive outcomes. In addition to false positives, hits arise by targeting undesired mechanisms. Compounds affect mechanisms other than differentiation, lipogenesis, or lipolysis, or can be toxic to preadipocytes, but not to fibroblasts.

A preferred primary screening protocol is as follows.
A. A high-throughput screen (HTS) technique for discovering compounds that modulate the differentiated preadipocyte or adipocyte's ability to take up and/or accumulate a fluorescently-labeled fatty acid (FA*) is featured. The assay features differentiated human preadipocytes, but the procedure can be modified in a number of ways to discover novel compounds affecting the adipocytes of human and other species (i.e., rodent). For example, the day of compound addition during preadipocyte differentiation process can be changed as well as the incubation period. Similarly, the day of FA* addition, its type of fluorescent label and chain length, and its concentration and incubation time can be modified.
B. Up to five cell strains of frozen primary human subcutaneous preadipocytes (e.g., subcutaneous preadipocytes) are used, but this number can be increased as needed. Omental and mesenteric preadipocytes can be used in a similar manner. Cells are 10 thawed in PM and grown in tissue culture flasks under standard incubation conditions (5% carbon dioxide, 37° C.). Cells are passaged 1:2 upon 80-100% confluency. Cells are passaged up to ten times to produce enough cells for primary screening.
C. Cells are seeded and differentiated, as described in the previous section, in 384-well plates.
D. On day 6 of preadipocyte differentiation, the DM is first changed as described in the previous section. Cells are then incubated in a replicate manner (i.e., in triplicate) with compounds. Compounds are typically dissolved 100% DMSO, but can be dissolved in a different solvent or aqueous solution. Compounds are further diluted in a PBS or similarly buffered solution in the absence or presence of albumin to aid compound solubility. After extensive mixing, the diluted compound is added to cells; for compounds initially dissolved in 100% DMSO, the final concentration of solvent in the cell solution is 0.1%.
E. Negative control cells are treated with the equivalent compound solvent or solution diluted in the compound diluent buffer (i e., for compounds dissolved in 100% DMSO and diluted in a PBS/BSA buffer, the negative control cells are treated in a similar manner without compound to give a final concentration of 0.1% DMSO). Positive control cells are treated with a compound or reagent known to modulate their ability to take up and/or accumulate fatty acids. For example, carbonyl cyanide p-(trifluoromethoxy)-phenylhydrazone (FCCP) is a potent uncoupler of oxidative phosphorylation in mitochondria, and inhibits FA* uptake and/or accumulation in a dose-responsive manner.
F. On day 9 of preadipocyte differentiation, FA* is diluted into a PBS or similarly buffered solution in the absence or presence of albumin to aid its solubility. See Table 11 for the current fatty acid buffer (FAB) used. The FA* currently used is 4,4-difluoro-5-methyl-4-bora-3a,4a-diaza-s-indacene-3-dodecanoic acid (C₁-BODIPY® 500/510 C₁₂, catalog # D-3823, Molecular Probes, Eugene, Oreg.). FA* stock concentration is 10 mM in 100% DMSO, frozen at −20° C.
G. Cell plates are pre-washed with FAB, preferably three times, to remove unincorporated compound, and then FA* is added in FAB at 10 uM final concentration. Cells are incubated for four hours at 5% carbon dioxide/37° C., then post-washed with FAB, preferably three times, to remove unincorporated FA*. Cellular fluorescence of triglyceride droplets that have incorporated FA* is measured on a microplate reader. Efficacy of compounds on inhibiting FA* accumulation is determined by:
   % Efficacy=100−(sample fluorescence/negative control fluorescence×100).
H. Subsequent determination of compound toxicity on cells is measured by incubating cells (after the post-wash and FA* reading) with an aliquot of Alamar Blue (catalog # DAL-1100, BioSource International), a fluorescent dye that is reduced via cellular metabolism. After incubation of cells with the dye for up to four hours, fluorescence of the reduced compound is read. Alternatively, compound toxicity on cells can be determined in an experiment separate from the primary screen uptake/accumulation assay. Toxicity of compounds is determined by:
   % Toxicity =100−(sample fluorescence/negative control fluorescence×100).

B2. Pathway and/or Target Identification

Secondary assays may be run on identified “hits” to define the pathway(s)/mechanism of action on which such an agent/hit may act. These pathway(s) comprise four categories: Adipogenesis (key transcription factors and enzymes that function in the differentiation of preadipocytes to adipocytes), lipogenesis (fatty acid uptake and/or triglyceride synthesis/storage), lipolysis (triglyceride breakdown), and oxidation (fatty acid metabolism). Preferably, only hits exhibiting acceptable toxicity limits are pursued. Pathways affected by the agents may be identified using assays well known to those of skill in the art.

Pathways related to adipogenesis may be investigated by measuring expression of related transcription factors such as those encoded by the PPAR gene family (e.g. PPARγ), and the C/EBP gene family (e.g. C/EBPα, C/EBPβ) and/or the transport protein GLUT4 gene and/or the aP2 gene. Such transcription factors and other proteins may be assayed using Western analysis using an appropriate antibody and extracts of cells as described by Ausubel and Brent; Short Protocols in Molecular Biology, 4^{th} Edition, 1999, John Wiley & Sons, Inc.). Preadipocytes or differentiated adipocytes may be exposed at various times to an agent in vehicle, then extracts made and run in a western blot assay. Anti-GLUT4 antibodies are available from Santa Cruz Biotech. Inc., (Santa Cruz, Calif.) and from Alpha Diagnostic International, (San Antonio, Tex.). PPAR antibodies are available from Research Diagnostics, Inc., Flanders, N.J.). C/EBP as well as PPAR antibodies are available from Active Motif, Carlsbad, Calif. Anti-aP2 antibodies may be obtained from Dr. D. A. Bernlohr at Univ. of MN. The reactive bands may be visualized using an enhanced chemoluminescence system (Amersham, Oakville, Ontario). Additionally, glycerol-3-phosphate dehydrogenase (G3PD) activity and/or PPAR gamma activation (e.g., Jeppesen et al, U.S. Pat. No. 6,468,996; Smith, U.S. Pat. No. 6,294,559) may be measured. G3PD activity may be measured as described by Sottile and Seuwen (2001) Analytical Biochemistry 293:124-128.

With respect to adipogenesis, adipocyte fat droplets are composed predominantly of triglycerides, which in turn are made up of fatty acid chains bound to a glycerol backbone. Active lipogenesis may be investigated by monitoring the conversion of radiolabeled glucose into the glycerol backbone, or of fatty acids such as palmitate or oleate into tri-, di-, and mono-glycerides; quantitation may be done via thin layer chromatography (TLC) to separate the lipid components, and subsequent scintillation counting of the desired TLC spot. Total triglyceride levels may be performed using commercially available kits, such as Triglyceride E kit from Wako (Osaka, Japan) or Infinity glycerol measurement kit from Sigma (St. Louis, Mo.), or by quantitating the amount of Oil Red O (Sigma, St. Louis, Mo.) staining of fat droplets in adipocytes.

Lipolysis may be investigated by measuring the release of glycerol or fatty acids from the fat droplets of adipocytes into the medium environment (eg. U.S. Pat. No. 6,096,338; U.S. Pat. No. 6,509,480). Glycerol may be measured using the techniques described above for Lipogenesis. Released fatty acids may be measured by preloading cellular triglycerides with a fluorescent fatty acid and then by monitoring differences in fluorescence under conditions favoring lipolysis. In addition, binding of fatty acids to ADIFAB (acrylodated intestinal fatty acid binding protein (ADIFAB); Molecular Probes, Eugene Oreg.) alter its fluorescence spectrum, which can be quantified to measure fatty acid release (Richieri, et al, 1992, J Biol Chem 267(33):23495-501; Richieri et al, 1994 J Biol Chem 269(39):23918-30; Richieri et al, 1999, Mol Cell Biochem 192(1-2):87-94).

Fatty acid oxidation in adipocytes may be investigated by monitoring the conversion of a tagged fatty acid (i.e., 14-C labeled fatty acid (i.e., oleate or palmitate)) to carbon dioxide in a closed system environment. In addition, oxygen consumption, reflective of cellular metabolism/oxidation, may be measured with an oxygen electrode such as a Biological Oxygen Monitor, MODEL 5300 (YSI Inc., Yellow Springs, Ohio, U.S.A.)). Alternatively, oxygen levels in the medium surrounding cells may be quantitated using an oxygen binding fluorescent probe (BD BioScience, Bedford, Mass.; Oxygen Biosensor System (OBS)).

Compounds having activity in one or more secondary screens (i.e., compounds effecting or acting via various pathways, may have particular usefulness based on the nature of the pathway. For example, compounds effecting adipogenesis (e.g., inhibiting differentiation) may have particular usefulness as therapeutics (or in the development of therapeutics) for use in pre-diabetic and/or post-liposuction control or treatment. Compounds effecting lipogenesis (e.g., inhibiting or decreasing triglyceride synthesis) may have particular usefulness as therapeutics (or in the development of therapeutics) for use in maintaining weight, for example, after weight loss. Compounds effecting lipolysis (e.g., increasing fat release) may have particular usefulness as therapeutics (or in the development of therapeutics) for use in reducing central fat. Compounds effecting oxidation (e.g., increasing fat burning) may have particular usefulness as therapeutics (or in the development of therapeutics) for use as diet aids, for example, during early weight loss or pre-gastric bypass.

D. Fat Depot-Specific Screens

Drugs that target visceral (mesenteric and omental) fat are of particular interest, since the accumulation of visceral fat (e.g., omental and/or mesenteric fat) carries a greater risk of morbidity and mortality than peripherally distributed fat (Rosenbaum, M., et al. (1997) New Engl. J. Med. 337:396-407). In particular, a high ratio of visceral to subcutaneous fat has been identified as a key risk factor for cardiovascular disease. Moreover, hypertrophy of visceral fat has been implicated in metabolic disorders, for example, the metabolic syndrome, Syndrome X.

Considerable evidence, both published and in preliminary studies, indicates that there are sufficient differences between subcutaneous and visceral preadipocytes to suggest that hits that primarily target one type of fat depot will be found. Significant differences exist in gene expression between the two cell types, glucocorticoid receptors and leptin, for example. Insulin action also appears to differ between omental and subcutaneous adipose tissue (Arner, P. (1997) Journal of Endocrinology 155:191-2), and, in a possibly related finding, preadipocytes from the two depots (from the same individuals) show a difference in susceptibility to the differentiation-promoting effects of PPARγ agonists.

C Lead Compound Characterization and/or Optimization

Lead compounds, also referred to herein as “hits”, exhibit biological activity detectable as an effect of fat cell, preadipocyte or adipocyte replication, differentiation or function. Preferably, lead compounds or “hits” exhibit a measurable or appreciable effect on replication, differentiation or function while being non-toxic to fat cells, preadipocytes or adipocytes. Lead compounds or “hits” preferably exhibit a potency of at least 500 nm, preferably at least 200 nM, more preferably at least 100 nM, more preferable at least 75 nM, even more preferably at least 50 nM, and even more preferably at least 10 nM. Fat depot specific lead compounds or hits preferably exhibit of at least 3-4-fold, more preferably at least 5-6-fold, more preferably at least 7-, S-, 9- or 10-fold.

IV. Pharmaceutical Compositions

Compounds identified according to the methodology of the instant invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Pharmaceutical compositions comprising compounds identified according to the methodology of the instant invention are particularly useful for the treatment of diseases and/or disorders including, but not limited to, obesity, diabetes, insulin-resistance, hyperinsulinemia, hyperglycemia, hyperlipidemia, weight regulation disorders, eating disorders, cachexia, anorexia and the like.

This invention is further illustrated by the following examples which should not be construed as limiting. The contents of all references, patents and published patent applications cited throughout this application are hereby incorporated by reference.

### EXAMPLES

### Example 1

### Isolation of High Yield, Essentially Pure Human Preadipocytes

This example describes methods of isolating a high yield, essentially pure population of human preadipocytes. The methods provided herein are suitable for the isolation of preadipocytes from subcutaneous, mesenteric and omental fat depots. At least six parameters of art-recognized adipocyte isolation procedures were varied and optimal conditions are described. Use of the optimized methods described herein results in reduced costs, time, and tissue requirements associated with human adipocyte screening assays. Currently, commercial isolation procedures for human preadipocytes require up to 70 g subcutaneous fat tissue (obtained by liposuction) to isolate 10⁶ cells. The preadipocyte source is further limited to only subcutaneous fat. Utilizing the methods disclosed herein, it is now possible to obtain 10⁶ cells from 1 g or less of human abdominal subcutaneous fat and further, to obtain such yields from both mesenteric and omental fat.

In isolating preadipocytes from fat tissue, fat tissue is transported to the laboratory, minced, digested in a collagenase solution, filtered, and plated. Each of these steps were varied to optimize the yield and quality of cultures. Yield was determined by calculating the number of preadipocytes recovered per gram of fat tissue, by counting cells at the differential replating step used to remove potentially contaminating cell types. Examples of parameters to be varied to enhance yield are presented in this example.

Decreasing Time Between Fat Tissue Harvest and Tissue Dissociation Enhances Preadipocyte Yield

Because fat cells are very fragile and have limited viability ex vivo, the effect on yield of isolating preadipocytes from fat tissue samples shortly after surgery, after 24 h, and after 7 d was determined. The fat samples were maintained at room temperature in a transport medium that contains essential nutrients and antibiotics. Preadipocyte yield decreased 22% within the first 24 hours following fat tissue harvest (53±4 vs. 42±4×10⁴ cells/g from fat tissue digested shortly after surgery and after 24 h, respectively; N=9; p=NS by Duncan's multiple range test). Within a week following fat tissue harvest, yield decreased 90% (53.1±4.2 vs. 5.3±0.6×10⁴ cells/g from fat tissue digested shortly after surgery and after 72 h, respectively; N=9; p<0.01 by Duncan's multiple range test). Overall, increasing time between surgery and digestion to isolate preadipocytes resulted in decreasing yield (p<0.005; ANOVA). Thus, while preadipocytes are more hardy than fat cells, presumably to permit fat tissue regeneration, yields are improved by rapid processing. Preferably, tissue dissociation (e.g., mincing and/or digestion) is accomplished within the first 24 hours following fat tissue harvest (e.g., surgery).

Modification of Mincing Technique Based on Fat Tissue Source Enhances Preadipocyte Yield

In comparing coarsely minced fat tissue to finely minced tissue before collagenase digestion, preadipocyte yield was higher in coarsely than finely minced abdominal subcutaneous (yield 120% higher) and mesenteric (80% higher) tissue. Conversely, yield was 7% higher in omental tissue that had been finely versus coarsely minced. Hence, modifications of this step in the preparation of preadipocytes have depot-specific effects on yield. This finding has been incorporated into the procedures used to isolate cells from different depots.

Modification of Media Components Enhances Preadipocyte Yield

Improved preadipocyte yield from human fat tissue has been found upon adding fetal bovine serum (FBS) to collagenase solution. Therefore, various types and concentrations of serum were used to optimize yield. Certain types of sera (e.g., Nuserum) improved yield, and this occurred in a depot-specific manner with the greatest improvements in yield achieved in omental tissue. Several serum components, such as albumin or anti-protease activity, could have contributed to this effect. Albumin and anti-protease effects were independently tested.

Bovine serum albumin (BSA) had a substantial effect on omental preadipocyte yield in particular. Yield was improved 1.5±0.2 fold from 91±16 to 123±16×10⁴ cells/g of omental fat tissue (p<0.02; paired T test; N=8). Interestingly, BSA had no 30 significant effect on abdominal subcutaneous or mesenteric preadipocyte yield, so that under these conditions, yield/g of tissue was higher from omental than subcutaneous or mesenteric fat (p<0.01; Duncan's multiple range test; N=8). Since more subcutaneous than omental fat is available from each patient, this approach for augmenting omental preadipocyte yield has enhanced the capacity to screen for agents with depot-specific effects.

Collagenase preparations contain some trypsin activity. To test if yield could be enhanced by augmenting the trypsin activity of collagenase, various amounts of trypsin were added to the collagenase solution. Trypsin supplementation caused a decrease in yield. Because of this and because serum, which enhances yield when added to collagenase inhibits trypsin activity, effects of adding trypsin inhibitors to the collagenase solution were determined. These agents caused a reduction in yield. Hence, an optimized collagenase solution including BSA rather than serum was developed.

Donor Characteristics have No Significant Effect on Preadipocyte Yield

Effects of donor characteristics on preadipocyte yield and digestion time were determined to assist in subject selection. In particular, body mass index (BMI; weight/height²), age, gender, and ethnicity on fat tissue digestion time and preadipocyte yield in mesenteric, omental, and abdominal subcutaneous fat specimens from 72 subjects was determined. Using applied regression analysis, it was determined that digestion time increased with donor age (p<0.05) and was longer for omental than subcutaneous, and subcutaneous than mesenteric fat (p<0.0001). However, host characteristics had no significant effect on preadipocyte yield.

Choice of Filter Enhances Preadipocyte Yield

After collagenase digestion, filters are used to remove undigested connective tissue. The effects of several different types of filters on yield were determined and it was found that gauze filters gave the best results. For example, 100 mesh nylon filters resulted in a 2.7 fold lower yield than gauze filters (p<0.0005; unpaired T test with pooled estimate of variance ; N=65).

Choice of Cell Culture Surface Enhances Yield (and Extent of Differentiation)

The effects of a number of plate types and plate coatings on yield and differentiation was determined. For example, the yield and/or extent of differentiation was reduced by the following products, compared to uncoated plates: poly-D-lysine, collagen type IV, and laminin. Based on these studies, optimal plates and coatings for yield and differentiation, were determined.

Optimization of parameters described in this example results in a greater than 3-fold improvement in yield as compared to previously published methods. In particular, it is possible to obtain one million cells from 1 g of fat tissue (or less). Moreover, these cells can be passaged at least 34 times before replicative arrest (noting fat-depot dependent differences) and the cells are further able to be maintained in the differentiated state for over 5 months (see below).

### Example 2

### Methods of Enhancing Human Preadipocyte Differentiation

The following example describes the optimization of methods used to differentiate human preadipocytes. The example details a thorough analyses of known and potential agents and conditions for promoting human preadipocyte differentiation. Experiments were performed with abdominal subcutaneous as well as mesenteric and omental preadipocytes. A variety of conditions were tested using the fatty acid-based cellulomic assay described in Example 4. Optimization of differentiation conditions enhances the usefulness of human preadipocytes in screening for modulators (e.g., inhibitors) of obesity.

TABLE 1

| Sample | Dex (M) | Uptake Ratio | % Diff. |
|---|---|---|---|
| Undiff. | 0 | 1 | 0 |
| Diff. | 0 | 28 | 50 |
| Diff. | 10⁻¹⁰ | 49 | 60 |
| Diff. | 10⁻⁷ | 79 | 70 |
| Diff. | 10⁻⁴ | 5 | 30 |

Variation of Dexamethasone Concentration to Optimize Differentiation

Table 1 shows effects of varying dexamethasone concentration on the ratio of fatty acid uptake by cells exposed to differentiation medium for 10 d to that by undifferentiated cells (signal-to-noise) and extent of differentiation (% cells containing doubly-refractile droplets evident by low power phase contrast microscopy). Briefly, human preadipocytes were cultured in either a control basal medium that does not promote differentiation (Undiff.) or a suboptimal differentiation medium (Diff.), to which various concentrations of dexamethasone (Dex.) were added for 10 d. The fatty acid uptake ratio was expressed as a function of fatty acid uptake in control medium. % Diff. represents the proportion of cells that developed doubly refractile lipid droplets visible by phase contrast microscopy by observers unaware of culture conditions. Means of 4 studies are shown. CoV was also determined (not shown) to monitor assay reliability.

Increasing Time in Differentiation Medium Results in Increasing Fatty Acid Uptake

The amount of time that preadipocytes were exposed to differentiation medium was investigated. Table 2 depicts the results of experiments in which preadipocytes were exposed to differentiation medium for the amount of time shown and the fluorescence was measured.

| | | | |
|---|---|---|---|
| Day | Fluor. | Avg | St. Dev. |

| | | | | |
|---|---|---|---|---|
| 0 | 733 | 725 | 729 | 8 |
| 3 | 715 | 710 | 713 | 5 |
| 0 | 708 | 696 | 702 | 12 |
| 7 | 725 | 727 | 726 | 2 |
| 0 | 690 | 675 | 683 | 15 |
| 11 | 654 | 662 | 658 | 8 |
| 0 | 696 | 723 | 710 | 27 |
| 15 | 684 | 705 | 695 | 21 |
| 0 | 744 | 748 | 746 | 4 |
| 19 | 690 | 686 | 688 | 4 |

Both the average fluorescence and the standard deviation are reported. Measuring both the average fluorescence and the standard deviation facilitates detection of signal, for example, in visceral preadipocytes which take longer to differentiate than abdominal subcutaneous preadipocytes. Increasing differentiation times further increased the signal-to-noise ratio in the fatty acid uptake assay.

Effects of Plating Density on Human Preadipocyte Differentiation

The effect of plating density was investigated. Plating density affected extent of differentiation (Table 3) and was optimized.

TABLE 3

| Plating | | | | | Morphological | Signal- |
|---|---|---|---|---|---|---|
| Density | Undifferentiated | CoV | Differentiated | CoV | Differentiation | to- |
| (cells/cm × 10⁻⁴) | (rfu: mean ± SEM) | (%) | (rfu: mean ± SEM) | (%) | (%) | noise |
| 1.5 | 753 ± 10 | 2.3 | 14166 ± 457 | 5.6 | 40 | 19 |
| 3.1 | 764 ± 14 | 3.2 | 16558 ± 831 | 8.7 | 60 | 22 |
| 4.7 | 771 ± 4.8 | 4.8 | 20249 ± 775 | 6.6 | 80 | 26 |
| 6.2 | 751 ± 3 | 0.7 | 23140 ± 513 | 3.8 | 90 | 31 |
| 9.4 | 940 ± 61 | 11.2 | 23560 ± 299 | 2.2 | 90 | 25 |

Increasing plating density was associated with an increase in the percent of lipid-containing cells and improving reliability and signal-to-noise ratios until densities of 6.2×10⁴ cells/cm² were surpassed. Hence, this plating density was used in subsequent analyses.

### Example 3

### Methods to Isolate and Differentiate Human Preadipocytes

The following example details an optimized method of isolating and differentiating human preadipocytes. This method allows for high yield of purified preadipocytes that have a high differentiative capacity.
1.) Prepare 1× Phosphate Buffered Saline/collagenase solution (3 mg collagenase/g of tissue and 1 ml 1× Phosphate Buffered Saline/mg collagenase)+3.5% fatty acid free BSA and filter.
2.) Remove tissue from transport bottle with sterile forceps.
   2A.) Omental tissue should be processed first since digestion time is longer.
      Omental tissue should be put into a sterile 100 mm dish and sectioned into approx. 5 g pieces.
      Each section of tissue should then be removed from the dish with sterile forceps and transferred to a 50 ml centrifuge tube that contains approx. 15 ml (3 ml/g of tissue) of the 1× Phosphate Buffered Saline/collagenase solution.
   2B.) The above procedure should be repeated for subcutaneous and mesenteric tissues.
3.) Mince the tissue in the solution to a fine consistency, be sure to use sharp sterile scissors.
4.) Vortex each tube thoroughly and put the tubes in the water bath to shake at 100 rpm and 37° C. Make sure the water bath is full enough so that all tissue is submerged.
5.) The tubes should be vortexed thoroughly every 5-10 minutes. The tubes should remain in the bath until all lumps appear dissolved but not so long that a clear fat supernatant layer appears.
6.) Once the solution appears homogeneous, each tube should be vortexed and filtered through a sterile funnel containing a double layered gauze filter. The samples should be filtered into a sterile 50 ml centrifuge tube.
7.) Centrifuge at 1000 rpm for 10 min.
8.) Gently aspirate off fatty layer and most of the supernatant leaving the pellet.
9.) Resuspend the pellet in 10 ml of Erythrocyte Lysing Buffer, vortex, and shake in water bath at 37° C. and 100 rpm for 5 min.
10.) Remove from water bath, vortex, and centrifuge at 1000 rpm for 10 min.
11.) Aspirate off most of the supernatant leaving the pellet.
12.) Resuspend the pellet in 10 ml of plating medium with 10% NuSerum, vortex, and plate overnight in 100 mm dishes. Store in incubator at 37° C. and 5% CO₂.
13.) After 24 hours, wash the cells three times with 10 ml of PBS/EDTA.
14.) After aspirating off the final wash and add 1 ml of the trypsin solution (5 ml of 10× trypsin+45 ml of PBS/EDTA) to the 100 mm dishes.
15.) Incubate the cells at 37° C. and 5% CO₂ until the majority of the cells have lifted.
16.) To each dish add 5 ml of plating medium with 10% NuSerum to inactivate the trypsin.
17.) Wash the cells from the dish with a narrow tip 5 ml pipette. Transfer the liquid cell suspension to a 50 ml centrifuge tube.
18.) Vortex and then centrifuge at 1000 rpm for 10 min.
19.) Aspirate off most of the supernatant and resuspend the pellet in plating medium with 10% NuSerum (5-10 ml depending on the size of the pellet).
20.) Transfer 100 μl from each tube to an epindorf tube. To each epindorf tube add 100 μt of Trypan blue and vortex. Count the cells using a hemocytometer, counting all four quadrants. # of cells=(cell count/4)×2×(1×10⁴) x (# ml cells are suspended in).
21.) Plate the cells on desired plates at a density of 5×10⁴ cells/cm² in plating medium with 10% NuSerum.
25. ) Incubate in plating medium with 10% NuSerum at 37° C. and 5%CO₂ until confluence is reached, changing media every 48 hours.
26.) At confluence remove plating medium and add either differentiating medium (to differentiate the cells), split the cells to passage them, or freeze the cells.

For differentiation, cells are seeded in culture dishes, flasks or plates at 1.5-6×10⁴ cells/cm², preferably 3×10⁴ cells/cm². Upon adherence, cells are 100% confluent, and PM is changed every two to four days until differentiation is begun, two to 14 days after seeding.

Cells are differentiated for the first three days in freshly prepared differentiation medium (DM) consisting of DMEM/F12, HEPES, sodium bicarbonate, penicillin, streptomycin, L-glutamine, transferrin, biotin, human insulin, panthothenic acid, fetuin, dexamethasone, triiodo-L-thyronine, rosiglitazone, and isobutylmethylxanthine (IBMX). Thereafter, IBMX may be reduced to 0-50% of the DM concentration.

On day 3 of differentiation, microscopic examination reveals that the cells have attained a rounded morphology. DM is changed on day 3 of differentiation to DM without IBMX (DM2).

On day 6 of differentiation, the cells are more rounded in morphology and have several tiny triglyceride droplets in their cytoplasm. DM is changed on day 6 of differentiation to DM without IBMX, rosiglitazone, insulin or dexamethasone (DM3). DM3 is changed thereafter every three days.

By day 9 of differentiation the triglyceride droplets are more numerous and larger in size. Further maintenance of cells in DM3 increases their triglyceride droplet size and number until the droplets eventually coalesce into one large droplet to displace the cell nucleus.

Solutions and Media

TABLE 4
Transport Medium

| Ingredients | Molarity | 500 ml | | 1 Liter | | Sigma# |
|---|---|---|---|---|---|---|
| 1X PBS | N/A | 500 | ml | 1 | L | Gibco |
| Gentamicin | N/A | 0.5 | ml | 1 | L | G-1397 |
| Amphotericin | N/A | 1 | ml | 2 | ml | A-2942 |
| Penicillin | 0.1 mM | 0.0295 | g | 0.059 | g | PEN-NA |
| Streptomycin | 0.06 mM | 0.05 | g | 0.10 | g | S-6501 |
| L-glutamine | 2 mM | 0.1461 | g | 0.2922 | g | G-5763 |

*pH to 7.4 using either HCl or NaOH and filter sterilize.
*The transport medium should be kept in either an amber bottle or wrapped in foil since the amphtericin is light sensitive and can become cytotoxic.

TABLE 5
Human Plating Medium with 10% NuSerum

| Ingredients | Molarity | 500 ml | | 1 Liter | | Sigma# |
|---|---|---|---|---|---|---|
| DMEM/F-12 | N/A | 450 | ml | 900 | ml | Gibco |
| Hepes | 13 mM | 1.78 | g | 3.90 | g | H-7006 |
| NaHCO₃ | 29 mM | 1.22 | g | 1.26 | g | S233-3 |
| Penicillin | 0.1 mM | 0.0295 | g | 0.059 | g | P-3032 |
| Streptomycin | 0.06 mM | 0.05 | g | 0.1 | g | S-6501 |
| L-glutamine | 2 mM | 0.1461 | g | 0.2922 | | G-8540 |
| dexamethasone* | 0.1 uM | 5 | ul of stock | 10 | ul of stock | D-4902 |
| Heat inactivated | N/A | 50 | ml | 100 | ml | BD |
| NuSerum | | | | | | BioSciences |
| | | | | | | #33-5504 |

*pH to 7.4 using either HCl or NaOH and filter sterilize.
*dexamethasone stock: Use 4 mg per 1 ml of ethanol

TABLE 6
Erythrocyte Lysing Buffer

| Ingredients | Molarity | | 300 ml | | 600 ml | | Sigma# |
|---|---|---|---|---|---|---|---|
| NH₄Cl | 154 | mM | 2.47 | g | 4.94 | g | A-0171 |
| KHCO₃ | 10 | mM | 0.3 | g | 0.6 | g | P-7682 |
| EDTA | <1 | mM | 0.011 | g | 0.022 | g | E-5134 |

*pH to 7.4 using either HCl or NaOH and filter sterilize.

TABLE 7
α-MEM10 Medium

| Ingredient | Concentration | 1000 ml | | Catalog # |
|---|---|---|---|---|
| water | N/A | 900 | ml | N/A |
| α-MEM | N/A | | | Gibco BRL |
| sodium bicarbonate | 26.8 mM | 2.25 | g | Fisher S233-3 |
| penicillin | 0.1 mM | 59 | mg | PEN-NA |
| streptomycin | 0.06 mM | 100 | mg | S-6501 |
| fetal bovine serum | N/A | 100 | ml | Gibco BRL |

- pH to 7.2 using hydrochloric acid and/or sodium hydroxide and filter-sterilize through 0.2 um membrane.

TABLE 8
Cell Washing Buffer

| Ingredient | Concentration | 500 ml | Catalog # |
|---|---|---|---|
| water | N/A | 450 ml | N/A |
| 10X PBS | N/A | 50 ml | Gibco BRL |
| EDTA | 0.7 mM | mg | E-5134 |

- pH to 7.4 using hydrochloric acid and/or sodium hydroxide and filter-sterilize through 0.2 um membrane.

TABLE 9
Trypsin Buffer

| Ingredient | Concentration | 500 ml | Catalog # |
|---|---|---|---|
| PBS/EDTA | N/A | 450 ml | see Table 5 |
| 10X Trypsin | 0.25X | 50 ml | Gibco BRL |

- pH to 7.4 using hydrochloric acid and/or sodium hydroxide and filter-sterilize through 0.2 um membrane.

TABLE 10
Plating Medium (PM)

| Ingredient | Concentration | 1000 ml | | Catalog # |
|---|---|---|---|---|
| water | N/A | 900 | ml | N/A |
| DMEM/F12 | N/A | 12.0 | g | Gibco BRL |
| HEPES, | 15 mM | 3.90 | g | H-7006 |
| sodium salt | | | | |
| sodium | 15 mM | 1.26 | g | Fisher S233-3 |
| bicarbonate | | | | |
| penicillin | 0.1 mM | 59 | mg | PEN-NA |
| streptomycin | 0.06 mM | 0.1 | g | S-6501 |
| L-glutamine | 2 mM | 0.2922 | g | G-5763 |
| dexamethasone | 0.1 μM | 10 | μl of stock* | D-4902 |
| NuSerum | N/A | 100 | ml | |

- pH to 7.2 using hydrochloric acid and/or sodium hydroxide and filter-sterilize through 0.2 um membrane.
*Dexamethasone stack is prepared fresh by dissolving 4 mg in 1 ml of 100% ethanol; vortex until dissolved.

TABLE 11
Addition Volumes of PBS/EDTA and Trypsin

| Culture Dish/Flask | ml PBS/EDTA | ml Trypsin |
|---|---|---|
| 100 mm dish | 2 | 1.5 |
| 25 cm² T-flask | 2 | 1 |
| 75 cm² T-flask | 3 | 3 |
| 150 cm² T-flask | 4 | 4 |
| 225 cm² T-flask | 5 | 5 |

TABLE 12
Human Adipocyte Differentiation (HAD) Medium (DM)

| Ingredients | Molarity | | 1 Liter | | Sigma # |
|---|---|---|---|---|---|
| water | N/A | | 900 | ml | N/A |
| DMEM/F12 | N/A | | 1 | package | Gibco |
| Powder | | | | | |
| Penicillin | 0.08 | mM | 0.0295 | g | PEN-NA |
| Streptomycin | 0.03 | mM | 0.050 | g | S-6051 |
| NAHCO₃ | 25 | mM | 1.26 | g | Fisher S233-3 |
| Hepes | 23 | mM | 3.90 | g | H-7006 |
| Transferin | N/A | | 0.010 | g | T-6549 |
| Biotin | 0.03 | mM | 0.0081 | | B-4639 |
| L-glutamine | 2 | mM | 0.2922 | | G-5736 |
| Human Insulin | 4 × 10⁻⁴ | mM | 0.00287 | g | I-2059 |
| Pantothenic Acid | 0.02 | mM | 0.00405 | g | P-5155 |

| | | | | |
|---|---|---|---|---|
| Fetuin | N/A | 1.0 | g | F-2379 |

| | |
|---|---|
| T3 (prepare 0.013 g in 10 ml; use 1 μl/L) | T-6397 |
| Dexamethasone (prepare 0.004 g in 1 ml ethanol; | D-4902 |
| use 10 μl/L) | |
| BRL/rosiglitizone (prepare 1 mg/280 μl DMSO; | |
| use 100 μl/L) | |

*For DM + IBMX add 1.2 × 10−6 g/ml
*pH before filtering to 7.4, use either HCl or NaOH

Plating medium (PM) may be changed on the third day post-seeding. The number of days cells are maintained post-confluency prior to differentiation may vary from 24 hours to 14 days, or beyond, with PM changes occurring every two to three days. The seeding density of preadipocytes may range from 1.5-6×10⁴ cells/cm².

### Example 4

### Development of a Fatty Acid-Based Celluomic Assay for Human Preadipocytes

This Example describes the development, optimization and validation of a fatty acid-based celluomic assay for human preadipocytes. The assay is useful for (1) detecting differences in the extent of adipogenesis of human preadipocytes, in particular, in small numbers of human preadipocytes. Such an assay is particularly useful in performing high-throughput screens for fat-modulatory agents as well as for optimizing conditions for culturing primary human adipocytes (see e.g., Examples 1-3, above). The assay is equally useful for monitoring the extent of differentiation or de-differentiation of human adipocytes.

To establish a rapid, 96 well format, automated assay of fatty acid uptake, the reproducibility and signal-to-noise ratios of four different fatty acid-fluorescent dye conjugates in undifferentiated and differentiated preadipocytes was determined. 3T3-L1 cells were used to compare the fluorescent dyes, the best of which was then selected for the human preadipocyte system. The fluorescent fatty acids tested were

1. 16-(9-anthroyloxy) palmitic acid (16-AP),
2. 12-(9-anthroyloxy) oleic acid (12-AO),
3. 4,4-difluoro-5-(2-thienyl) 1-4-bora-3a,4a-diaza-s-indacene-3-dodecanoic acid (BODIPY 558/568 C12), and
4. 4,4-difluoro-5-methyl-4-bora-3a,4a-diaza-s-indacene-3-dodecanoic acid (BODIPY 500/510 C12).

These fatty acid-dye conjugates were chosen because their final size is close to that of physiologically relevant C:18 fatty acids, the most abundant in human fat cells and in the diet (Kokatnur, M., et al.(1979) Am. J. Clin. Nutr. 32:2198-205; Llado, I., et al. (1996) Biochem. Mol. Biol. Int. 40:295-303)). Furthermore, these dyes are vital, allowing the cells to be re-used in additional assays. 3T3-L1 cells were seeded in the wells of a 96-well plate at a density of 3.3×10³ cells/cm² (1×10³ cells/well). After reaching confluence, cells were exposed to differentiation medium for 0 and 9 days. The wells were washed with assay buffer (150 mM NAZI, 10 mM NaPO₄; 3 mM KCl; 10 μM CaCl₂; 1 mM MgCl₂; 25 mM glucose; pH 7.4) containing 20 μM fatty acid-free BSA, pre-warmed to 37° C. The fluorescent fatty acids were added in assay buffer to a final concentration of 10 μM, and incubated at 37° C. for one hour. Wells were washed with assay buffer containing 0.1% BSA. Results are shown in Table 13.

TABLE 13
Comparison of fluorescent fatty acids for uptake by 3T3-L1 cells.
Results represent means ± SEM

Raw Fluorescent Units

| Fluor. Dye | Day 0 | Day 9 |
|---|---|---|
| 12-AO | 3613 ± 160 | 10960 ± 1172 |
| 16-AP | 2944 ± 404 | 8528 ± 20 |
| BODIPY 500/510 C12 | 404 ± 21 | 56869 ± 2093 |
| BODIPY 558/568 C12 | 116 ± 9 | 3622 ± 641 |

Day 0: Cells were not incubated in differentiation medium.
Day 9: Cells were incubated 9 days in differentiation medium prior to the lipid uptake assay.

BODIPY 500/510 C12 dye had the best signal-to-noise ratio of all the fluorescent fatty acids tested. Therefore, this dye was chosen for further investigation. In separate experiments, the dye was tested at different time points for uptake by the 3T3-L1 cells.

TABLE 14
BODIPY uptake increases with incubation time. 3T3-L1 cells were
incubated with 10 μM BODIPY 500/510 C12 for the times indicated.

| Time | Day 0 | Day 8 | St. Dev. |
|---|---|---|---|
| 10 min | 1290.263 | 1198.325 | 1525.697 |
| 30 min | 510.3125 | 18518.67 | 118.4417 |
| 120 min | 686.9375 | 46363 | 107.1977 |
| 240 min | 1104.125 | 49153.33 | 329.7533 |

Means of triplicates ± SEM are shown.
rfu: raw fluorescent units.

The results shown in Table 14 indicate that the minimum time required for optimum assay results is between 30 and 120 minutes with 10 FM BODIPY 500/510 C 12. The dye was also tested for uptake at various concentrations, and 10 μM was the concentration that gave the best results. The data presented in Table 14 also demonstrate the excellent signal-to-noise ratio (day 8 vs. day 0) for cells that were incubated with dye for 120 min. or more.

Table 15 indicates the results of experiments in which 3T3-L1 cells were exposed to a differentiation inducing medium from day 0. Fatty acid uptake was assayed by exposing cells to BODPY for 120 minutes at each of the times represented. The results indicate that the longer a preadipocyte is exposed to differentiation medium (i.e., the more differentiated the cell is) the more dye uptake is observed.

TABLE 15
The Effect of Differentiation on Fatty Acid Uptake

| Day | Fluor. Units | St. Dev. |
|---|---|---|
| 0 | 1166.67 | 196.869 |
| 2 | 5006.75 | 1144.76 |
| 4 | 10884.5 | 3932.8 |
| 6 | 15441.8 | 3011.79 |
| 8 | 18685 | 4491.85 |
| 10 | 31645.8 | 12338.2 |
| 12 | 37910 | 11694.7 |

The use of 3T3-L1 cells in the development of the lipid uptake assay in a high-throughput format facilitated the development of an assay using human preadipocytes.

The BODIPY 500/510 C12 fatty acid uptake assay was optimized in the human preadipocyte system. The reliability of the assay was increased substantially. When the methods developed in 3T3-L1 cells were first used in human preadipocytes, the intra-assay standard coefficient of variation (CoV) was 40%. Once assay methods were optimized the CoV was reduced to 3.8%. To achieve this improvement in reliability, a variety of culture plates, dye incubation times, washing conditions, fluorimetric settings, plating densities, and times between induction of differentiation and assay were tested. By increasing plating density to 6.2×10⁴ cells/cm², the CoV was reduced, the extent of differentiation increased, and the signal-to-noise ratio enhanced. By increasing incubation time, both reliability and signal-to-noise ratio were further increased.

### Example 5

### Verification of Fatty Acid Uptake Assay as an Indicator of Adipogenic Differentiation and Verification of Improved Human Adipocyte Differentiation Methods

Abdominal subcutaneous preadipocytes were prepared as previously described and parallel, fourth-passage cultures were treated for 10 days with the Human Adipocyte Differentiation (HAD) medium of Table 12. Control cells were differentiated according to a published protocol or were cultured in a medium that does not promote lipid accumulation. The cells were assayed for fluorescent fatty acid assay uptake, as described herein. Using the published differentiation method, even with isobutylmethylxanthine (IBMX) treatment from days 1 to 3 (which further improves differentiation), only an 8 fold increase in uptake occurred compared to undifferentiated control cells (CoV=18%; N=4). In parallel cultures treated using HAD medium, the increase was 129 fold (CoV=1%). These conditions were also tested in G3PD and aP2 secondary assays as follows.

Gycerol-3-phosphate dehydrogenase (G3PD) activity assay. G3PD expression increases midway through adipogenesis and the G3PD promoter is activated by both C/EBPα and PPARγ, reflecting activity of these key adipogenic pathways. G3PD activity was measured as described by Sottile and Seuwen (2001) Analytical Biochemistry 293:124-128. G3PD may be measured in supernatants of cell homogenates by following NADH disappearance spectrophotometrically. The assay is simple and can be done using relatively small numbers of cells. Preferred assay parameters are as follows: CoV<1.6%; signal-to-noise 34-fold; minimal detectable limit: 0.05 units (nmole dihydroxyacetone phosphate/ml×min); minimal detectable difference: 0.019 units; activity in undifferentiated human preadipocytes: 5.4 units/10⁶ cells).

Western assay for aP2 and competimer rtPCR assay for PPARγ. Several different antibodies and primer sets were tested and it was decided to use Western assays for aP2 and competimer rtPCR for PPARγ for monitoring differentiation of preadipocytes. aP2 expression is very specific for adipose cells, undergoes a large increase during differentiation, and is fat depot-dependent. The aP2 Western antibody was sensitive and provided a linear response.

G3PD activity in human adipocytes cultured in the HAD medium of Table 10 was 184.0 units. In adipocytes cultured according to the published method, activity was 9.3 units. In control undifferentiated cultures, activity was 5.4 units. aP2 protein was also more abundant in the cells differentiated with HAD medium.

Thus, the differentiation methodology of the instant invention results in enhanced differentiation even when using subcultured (i.e., fourth-passage) cells, thus greatly reducing costs and the number of fat samples required. The differentiation methodology of the instant invention also produces differentiated preadipocytes more rapidly than published methods, saving time, medium changes, and associated expense and contamination risk.

### Example 6

### HTS for Compounds that Inhibit FA* Accumulation in Human Subcutaneous Adipocytes

Having optimized the fatty acid uptake assay using rodent and human adipocytes and verified that the assay is a true indicator of phenotypic differentiation of adipocytes, high-throughput screening assays were developed featuring human preadipocytes isolated and cultured according to the present invention.

Primary screens for inhibition of fatty acid uptake are carried out using human subcutaneous preadipocytes in each of two assay formats. In a first assay format, cells are exposed to natural product and synthetic library components early in the differentiation process (days 1-3 following addition of differentiation medium). In a second assay format, cells are exposed to library components late in the process (days 7-10 following addition of differentiation medium), after at least 80% of cells have differentiated.

Subcutaneous human preadipocytes (passage 3-5) are seeded in plating medium at 3.0×10⁴ cells/cm² in 96-well or 384-well plates, giving 100% confluence upon adherence. Plating medium is exchanged 48- to 120-hours later for differentiation medium plus IBMX to initiate adipocyte differentiation. After 72 hours, differentiation medium containing a lower concentration of IBMX is used. This medium is changed thereafter every three to four days until cells are assayed for fluorescent fatty acid (FA*) accumulation.

Cells are treated in triplicate with compounds at various concentrations. Compounds are first diluted in a phosphate-buffered saline solution containing 0.1% fatty-acid free bovine serum albumin—and then added to cells. Negative control cells are treated with DMSO, the solvent used for initially dissolving compounds, at 0.1% final concentration. Positive control cells are treated with carbonyl cyanide p-(trifluoromethoxy)-phenylhydrazone (FCCP, a potent uncoupler of oxidative phosphorylation in mitochondria). Cells are then allowed to differentiate for 72 hours more before being assayed for FA* accumulation as follows.

Differentiated adipocytes are pre-washed three times with a fatty acid buffer (FAB) containing: DMEM/F12 (1:1), 15 mM HEPES, 15 mM NaHCO3, and 2 mM glutamine. Cells are then incubated with FA* at 10 μM in FAB plus 0. 1% fatty-acid free BSA (FAB+) for 4 hours at 37° C. Cells are then post-washed five times with FAB+ and FA* accumulation is measured on a microplate reader at excitation/emission 485/635 nm.
% efficacy of compound on inhibiting FA* accumulation is calculated as follows:
% Efficacy=100−(sample fluorescence/negative control fluorescence×100).

Subsequent determination of compound toxicity is measured by incubating cells with one tenth of total cell volume of Alamar Blue (an indicator of cellular metabolism) for up to four hours before measuring fluorescence of the reduced compound. Cell survival is measured by monitoring the fluorometric change produced in the dye upon its reduction by living cells (Fields, R. et al. (1993) Am. Biotechnol. Lab. 11:48-50).

Toxicity is calculated as follows:
% Toxicity=100−(sample fluorescence/negative control fluorescence×100).

Table 14 depicts the results of screening various concentrations of FCCP for both inhibition of FA* accumulation and cellular toxicity in differentiating adipocytes.

TABLE 14

FCCP:

| | 0 μM | 0.3 μM | 1 μM | 3 μM |
|---|---|---|---|---|
| % Efficacy: | 0 | 42 | 79 | 90 |
| % Toxicity: | 0 | −1 | −4 | 5 |

In a first round of screening, 50,000 combinatorial compounds and 10,000 natural product compounds obtained from microbial extracts were screened for efficacy. Primary hits were selected as those producing a greater than 85% decrease in fatty acid content as compared to controls. This screening is described in detail in U.S. application Ser. No. 10/201,588, which is incorporated herein by reference. Primary hits were evaluated further to determine concentration response relationships for efficacy and toxicity. Natural compound hits resulted in a greater than 80% decrease in fatty acid content without any observable toxicity to human cells (adipocytes and fibroblasts). Combinatorial compound hits show comparable efficacy at low nanomolar levels without toxicity. Using these assays, multiple, non-toxic, chemically dissimilar hits have been identified.

Hits are further characterized using secondary screens to evaluate the biochemical mechanism of action and identify molecular targets or pathways effected by the hit. Compounds affecting accumulation of fatty acid may act via a variety of relevant mechanisms such as decreasing fatty acid uptake and/or triglyceride synthesis/storage (lipogenesis), or increasing triglyceride breakdown (lipolysis) and/or fatty acid oxidation. In addition, hits are evaluated for their effects on the differentiation process and, optionally, are evaluated for their specificity toward adipocytes from the distinct anatomical depots (see Example 10).

### Example 7

### Identification of Agent for Use in Fatty Acid Accumulation/Uptake Modulation

Preadipocytes were isolated from human subcutaneous fat tissue and cultured as described previously. Cells were seeded in a 384-well plate at 3×10⁴ cells/cm² (giving 100% confluency upon adherence) and maintained at 100% confluency for five days in 50 μl of Plating Medium (PM), then differentiated.

Day 0 of Differentiation: Plating medium was completely exchanged with 50 μl freshly prepared Differentiation Medium (DM), described in Example 3 including 540 micromolar IBMX.

Day 3 of Differentiation: 40 μl of the medium ( 4/5 of total volume) was exchanged with DM minus IBMX (DM2).

Day 6 of Differentiation: All 50 I¹ of DM2 was exchanged with DM minus IBMX, rosiglitazone, insulin and dexamethasone (DM3). A test agent was added to cells at various concentrations (1000, 300, 100, 30 and 10 nM).

Day 9 of Differentiation: To remove unincorporated test agent, the cell plate was pre-washed three times in Fatty Acid Buffer containing 0. 1% fatty acid-free BSA (FAB). Fluorescent fatty acid (FA*, D-3823, Molecular Probes, Eugene Oreg., 10 mM stock solution in DMSO, stored at -20° C.) was diluted in FAB, then added to cells at 5 μM concentration.

Cells were incubated for four hours at 5% carbon dioxide/37° C., after which the plate was post-washed three times in a manner similar to the pre-wash.

Fluorescence of FA* incorporated into adipocytes was determined with a microplate reader. The % efficacy of the test agent on inhibiting FA* uptake and/or accumulation was determined as described previously.

To determine compound toxicity toward cells, the plate then was incubated for three hours more at 5% carbon dioxide/37° C. after 5 μl ( 1/10) addition of Alamar Blue (BioSource International, Inc.) to the sample wells. Fluorescence of reduced Alamar Blue was determined with a microplate reader. The % toxicity of the test agent was determined as described previously.

Adipogenesis

The test agent was assayed for its effect on expression of G3PD, an indicator of the extent of adipogenesis in differentiated preadipocytes. G3PD may be measured by monitoring NADH disappearance spectrophotometrically as previously described. Preadipocytes were isolated from human subcutaneous fat tissue and cultured as described previously. Cells were seeded in a 96-well plate at 3×10⁴ cells/cm² and maintained at 100% confluency for five days in 200 μl of Plating Medium (PM). Preadipocytes were differentiated as described above in the presence of the agent, except that volume changes were 200 μl on Day 0 of Differentiation and 160 μL on Day 3 of Differentiation.

Day 6 of Differentiation: All 200 μl of DM was exchanged with DM minus IBMX, rosiglitazone, insulin and dexamethasone. The test agent was added to cells at 30 μM final concentration (0.1% DMSO). 1 nM of human tumor necrosis factor alpha (TNFα, a cytokine that inhibits preadipocyte differentiation and adipogenesis) also was added to cells in a similar manner as a positive control. 0.1% DMSO also was added to cells as a negative control.

Day 9 of Differentiation: Treated cells were pre-washed three times with Fatty Acid Buffer (FAB, described in previous section) with 160 μl volume exchanges to remove the DM and compound. Cells were incubated for four hours at 5% carbon dioxide/37° C. (under conditions described above), then assayed for G3PD activity.

Lipogenesis

The test agent was assayed for its ability to modulate triglyceride synthesis in adipocytes, as monitored by 14-carbon labeled glucose conversion to the glycerol backbone of triglycerides. Preadipocytes were isolated from human subcutaneous fat tissue and cultured as described previously. Cells were seeded in a 12-well plate at 3×10⁴ cells/cm² and maintained at 100% confluency for five days in 2 ml of PM. Preadipocytes were differentiated as described above, except that volume changes were 2 ml on Day 0 Differentiation and 1.6 ml on Day 3 Differentiation.

Day 6 Differentiation: All 2 ml of DM was exchanged with DM minus IBMX, rosiglitazone, insulin and dexamethasone. The test agent was added to cells at 30 μM final concentration (0.1% DMSO). 10 μM wortmannin (a phosphoinositide-3 kinase inhibitor that affects glucose metabolism in cells) also was added to cells in a similar manner as a positive control. 0.1% DMSO also was added to cells as a negative control. Day 9 Differentiation: Treated cells were pre-washed once with 2 ml of Lipogenesis Buffer (described below), pre-warmed to 37° C. This volume was then exchanged with 2 ml of Lipogenesis Buffer plus 10 nM insulin and.10 μM uniformly labeled 14-carbon glucose. The cell plate was incubated for four hours at 5% carbon dioxide/37° C., then the treated cell wells were post-washed twice with Lipogenesis Buffer, then placed on ice.

| Ingredient | Concentration | 1000 ml | | Catalog # |
|---|---|---|---|---|
| water | N/A | 900 | ml | N/A |
| DMEM* | N/A | 12.0 | g | Gibco BRL |
| HEPES, sodium salt | 15 mM | 3.90 | g | H-7006 |
| sodium bicarbonate | 15 mM | 1.26 | g | Fisher S233-3 |
| L-glutamine | 2 mM | 0.2922 | g | G-5763 |
| glucose | 5 mM | 0.900 | g | |
| fatty acid-free BSA | 0.1% | 1.0 | g | Intergen 3320-01 |

pH to 7.2 using hydrochloric acid and/or sodium hydroxide and filter-sterilize through 0.2 um membrane.
*DMEM minus glucose

Lipid Extraction: All of Lipogenesis Buffer was removed from the treated cell well, and 200 ul of ice-cold 1× trypsin solution was added. After a few minutes of incubation, cells were detached from the well with a cell scraper and transferred to a glass test tube on ice. 175 ×l of ice-cold 21.4 mM hydrochloric acid were added to the well and pipeted up and down several times to remove any residual cells attached; the contents were transferred to the same test tube on ice. 1.5 ml of chloroform:methanol (2:1) was added to the test tube and vortexed to extract lipids into the organic phase. The test tube was centrifuged at 400×g for five minutes at room temperature to separate the organic and aqueous phases. The lower organic layer was removed with a glass pipet and transferred to a new screwtop glass test tube. The contents were dried down under nitrogen gas and stored at −20° C. until thin layer chromatography (TLC) separation.

TLC: The lipid film was redissolved in 50 μl of chloroform, and 5 μl of the solution were transferred to a scintillation vial for 14-carbon quantitation. Another 5 μl were spotted on Baker Silica Gel TLC plates (plastic backing), and then run in a chamber with chloroform:diethyl ether:acetic acid. The lipid spots were visualized with gaseous iodine, and the triglyceride spot was cut out and placed in a scintillation vial for 14-carbon quantitation.

Lipolysis

The test agent was assayed for its ability to modulate lipolysis in adipocytes, as monitored by release of pre-loaded fluorescent fatty acid (FA*) from triglyceride droplets in the cells. Preadipocytes were isolated from human subcutaneous fat tissue and cultured as described previously. Cells were seeded in a 384-well plate at 3×10⁴ cells/cm² and maintained at 100% confluency for five days in 50 ul of PM. Preadipocytes were differentiated as described above.

Day 8 Differentiation: FA* (D-3823, Molecular Probes, Eugene Oreg.) was diluted in FAB and added to cells at 10 μM.

Day 9 Differentiation: Treated cells were pre-washed three times with FAB to remove unincorporated FA*. Fluorescence of loaded FA* in the triglyceride droplets of cells was measured with a microplate reader The test agent was added to a portion of the cells (1000, 300, 100, 30 and 10 nM). Forskolin (an inhibitor of adenylate cyclase) was added to cells in the micromolar range in a similar manner as a positive control. 0.1% DMSO was added to cells as a negative control. Cells were incubated for four hours at 5% carbon dioxide/37° C. Cells then were post-washed three times with FAB to remove released FA*, and fluorescence was measured. The ratio of FA* fluorescence before and after compound exposure was determined to quantitate the extent of lipolysis.

The test agent was shown to inhibit lipolysis.

Oxidation

The test agent was assayed for its ability to modulate oxygen consumption in adipocytes, as monitored by quantitation of oxygen levels in the medium surrounding cells using an oxygen binding fluorescent probe in a 96-well round bottom plate (BD BioScience Oxygen Biosensor System (OBS)). Preadipocytes were isolated from human subcutaneous fat tissue and cultured as described previously. Cells were seeded in a 6-well plate at 3×10⁴ cells/cm² and maintained at 100% confluency for five days in 3 ml of PM. Preadipocytes were differentiated as described, except that volume changes were 3 ml on Day 0 Differentiation and 2.4 ml on Day 3 Differentiation.

Day 6 Differentiation: All 3 ml of DM were-exchanged with DM minus IBMX, rosiglitazone, insulin and dexamethasone. The test compound was added to cells at 30 μM final concentration (0.1% DMSO). 10 ,μM carbonyl cyanide p-(trifluoromethoxy)-phenylhydrazone (FCCP, a potent uncoupler of oxidative phosphorylation in mitochondria) also was added to cells in a similar manner as a positive control. 0.1% DMSO also was added to cells as a negative control.

Day 9 Differentiation: Since the 96-well OBS plate has the oxygen-sensitive fluorescent probe attached in a silica matrix to the round-bottom well, adherent cells cannot attach and grow well. Therefore, to use this system the differentiated preadipocytes must be detached from the 6-well plate and placed in the OBS plate in suspension.

Treated cells in the 6-well plate were pre-washed once with 2 ml of PBS/EDTA (see previous section for recipe), then incubated with 250 μl of 1× trypsin in PBS/EDTA for five minutes, with swirling every 30 seconds. 500 μl of PM minus penicillin and streptomycin, and also minus phenol red (MediaTech 90-090-PC; the phenol red color can interfere with the OBS probe fluorescence) was added, and the cells were gently detached with pipetting. More PM was added such that transferring 200 μl into an OBS well gave 25,000 cells/well.

Cells in the 6-well plate not treated with compounds also were detached and placed in OBS wells in a similar manner. These cells then were treated with the test agent (30 μM), FCCP (1 μM) or DMSO (0.1%) for acute exposure, to compare to cells treated for three days. Empty OBS wells also were treated with water or 100 mM sodium sulfite to demonstrate the full range of probe fluorescence (sodium sulfite will remove all oxygen from the system to allow complete probe fluorescence).

After the addition of cells, compounds and sodium sulfite, the OBS plate fluorescence was determined with a microplate reader. After incubation for four hours at 5% carbon dioxide/37° C., the plate was read again. The change in fluorescence was used to quantitate the amount of oxygen consumption in the medium surrounding the cells.

### Example 8

### Animal Studies

The agent from Example 7 was tested for its ability to inhibit weight gain in Sprauge-Dawley rats fed a commercial high fat diet (Harlan Teklad, Product No. TD 98211). Weight gain was significantly less in rats injected with the agent as compared to controls.

### Example 9

### Effects of Fat Depot Origin on Human Preadipocyte Replication, Differentiation, and Fatty Acid Handling

The following example describes the effects of fat depot origin on preadipocyte replication, differentiation and fatty acid handling. The experiments show that preadipocytes from the three different fat depots respond differently to differentiation as evident through differential expression of differentiation markers. The example also provides methodology that allows for detection of substantial differences in characteristics of preadipocytes from different regions.

Using rat preadipocytes, it was determined that regional differences in expression of fatty acid binding proteins (aP2 and keratinocyte lipid binding protein), as well as enzymes of fatty acid metabolism (including carnitine palmitoyl transferase I and G3PD) contribute to interdepot variation in fatty acid uptake and esterification. Regional differences in cultured preadipocyte aP2 levels were reflected in regional differences in fat cell aP2 expression iii vivo. Interdepot differences were found in human preadipocyte aP2 expression and G3PD activity as well. These likely contribute to interdepot differences in fatty acid handling and are related to variation in capacity for differentiation. aP2 was highest in differentiating human abdominal subcutaneous preadipocytes, lower in mesenteric preadipocytes, and lowest in omental preadipocytes as shown by a western blot using a aP2 specific antibody after 10 days. Even in omental preadipocytes, aP2 expression increased with differentiation, particularly in primary culture.

After 10 days of treatment with differentiation medium, abdominal subcutaneous preadipocyte G3PD activity was 235.2 units/million cells, mesenteric was 28.2, and omental 17.2, while undifferentiated control cell G3PD was 5.4. Others have also found interdepot variation in human preadipocyte G3PD activity (Hauner, H., et al. (1991) Int. J. Obesity 15:121-6)). After longer periods of differentiation medium exposure, G3PD activity increased. Hence, even after relatively short periods of exposure to differentiation inducing media, distinct patterns of fatty acid binding protein expression and lipogenic enzyme activities occur in preadipocytes cultured from different regions of the same individual, indicating the feasibility of developing treatments that have differential effects on fat tissue from different regions.

Others have reported the PPARγ expression does not differ among human subcutaneous and omental preadipocytes even though they did find that thiazolidinediones, which are PPARγ activating ligands, caused more extensive differentiation of subcutaneous than omental cells (Adams, M., et al. (1997) J. Clin. Invest. 100:3149-53)). By contrast, subcutaneous, omental, and mesenteric preadipocytes differentiated according to the methodology described herein exhibited significant differences in both PPARγ2 mRNA and protein abundance. Moreover, interdepot differences in human preadipocyte cytokine release (previously reported in vivo) have been found. Particularly in the case of TNFα, these differences in preadipocyte autocrine factor release may have important effects on regional patterns of fat tissue function. TNFα, for example, affects differentiation and lipid accumulation, as well as activities of key adipogenic transcription factors including PPARγ and C/EBPα (Ron, D., et al. (1992) J. Clin. Invest. 89:223-33; Stephens, J. M., et al (1992) J. Biol. Chem. 267:135804; Williams, P. M., et al. (1992) Mol. Endocrinol. 6:1135-41; Zhang, B., et al. (1996) Mol. Endocrinol. 10: 1457-66; Szalkowski, D., et al. (1995) Endocrinology 135:1474-81)). Thus, multiple regulatory pathways important in controlling lipid accumulation differ among fat depots, and these differences in tissue characteristics are reflected in preadipocytes cultured according to the instant methodologies.

### Example 10

### Fat Depot-Specific Screening Assays

The following example provides methods to identify compounds (e.g., small molecules, peptides, or peptidomimetics) that are capable of modulating the proliferation and/or growth of human adipocytes. Primary screens identify compounds that are capable of combating obesity by reducing visceral fat. Those compounds are then tested with omental and mesenteric cells to determine their ability to modulate preadipocytes from these depots.

Subcutaneous preadipocytes, which are more easily obtained in the quantities necessary, are used for the primary screen. Because the reduction of visceral fat is important for a drug designed to combat obesity, omental and mesenteric preadipocytes are used in secondary screens. The secondary screens may also identify compounds that have a greater activity on omental or mesenteric than on subcutaneous preadipocytes. This information is important in the prioritization of compounds for further study.

The accumulation of visceral fat is more strongly associated with obesity-related diseases, such as diabetes, than is the accumulation of fat in other depots. Therefore, potential drugs aimed at decreasing obesity should at the very least target all fat depots and, at best, target preferentially visceral fat. The primary screens for inhibition of fatty acid uptake are carried out as described in Example 8 in either of the two assay formats. The secondary screens for inhibition of fatty acid uptake are similar to the primary screens, but utilize omental and mesenteric preadipocytes. Therefore, omental and mesenteric cells are exposed to hits from the primary screens using the two assay format involving early and late addition of library components.

Equivalents

Those skilled in the art will recognize, or be able to ascertain using no more than routine experimentation, many equivalents to the specific embodiments of the invention described herein. Such equivalents are intended to be encompassed by the following claims.

1. A method for identifying a fat modulatory compound, comprising:
(a) obtaining a high-yield, essentially pure human preadipocyte population;
(b) culturing said preadipocyte population under conditions sufficient to induce differentiation;
(c) contacting said population with a test compound at least 1-3 days following initial culture in the differentiation medium;
(d) maintaining said population in the presence of said test compound; and
(e) assaying the population for fatty-acid uptake or accumulation, a detectable fluctuation in which indicates that the test compound is a fat modulatory compound.

2. A method for identifying a fat modulatory compound, comprising:
(a) obtaining a high-yield, essentially pure human preadipocyte population;
(b) culturing said preadipocyte population under conditions sufficient to induce differentiation;
(c) contacting said population with a test compound at least 6-10 days following initial culture in the differentiation medium;
(d) maintaining said population in the presence of said test compound; and
(e) assaying the population for fatty-acid uptake or accumulation, a detectable fluctuation in which indicates that the test compound is a fat modulatory compound.

3. A method for identifying a potential weight loss or anti-obesity agent, comprising:
(a) obtaining a high-yield, essentially pure human preadipocyte population;
(b) culturing said preadipocyte population under conditions sufficient to induce differentiation;
(c) contacting said population with a test compound at least 1-3 days following initial culture in the differentiation medium;
(d) maintaining said population in the presence of said test compound; and
(e) assaying the population for fatty-acid uptake or accumulation, a detectable inhibition of which identifies the test compound as a potential weight loss or anti-obesity agent.

4. A method for identifying a potential weight loss or anti-obesity agent, comprising:
(a) obtaining a high-yield, essentially pure human preadipocyte population;
(b) culturing said preadipocyte population under conditions sufficient to induce differentiation;
(c) contacting said population with a test compound at least 6-10 days following initial culture in the differentiation medium;
(d) maintaining said population in the presence of said test compound; and
(e) assaying the population for fatty-acid uptake or accumulation, a detectable inhibition of which identifies the test compound as a potential weight loss or anti-obesity agent.

5. The method of claim 1, further comprising the step of determining the effect of the test compound on at least one of adipogenesis, lipogenesis, lipolysis, and oxidation.

6. The method of claim 3, further comprising the step of determining the effect of the test compound on at least one of lipogenesis, lipolysis, and oxidation in the presence of said compound,wherein the effect is inhibition of lipogenesis, increase in lipolysis or increase in oxidation.

7. The method of claim 1, wherein the conditions sufficient to induce differentiation comprise culturing said population in a serum-free differentiation medium.

8. The method of claim 1, further comprising the steps of determining the toxicity of said test compound on said human preadipocyte population.

9. The method of claim 1 further comprising the step of determining the toxicity of said test compound on a control cell population.

10. A method of identifying a potential weight loss or anti-obesity agent comprising:
(a) selecting a fat modulatory compound identified according to claim 1, said compound being capable of inhibiting fatty-acid uptake or accumulation in the absence of substantial toxicity to said adipocyte population; and
(b) determining the effect of the test compound on at least one of lipogenesis, lipolysis, and oxidation in the presence of said compound,
wherein the effect is inhibition of lipogenesis, increase in lipolysis or increase in oxidation identifies said compound as a potential weight loss or anti-obesity agent.

11. The method of claim 10, further comprising the step of determining the effect of the test compound on adipogenesis.

12. A method for identifying a target for an agent modulating weight gain comprising:
identifying a compound that inhibits differentiation of preadipocytes to adipocytes in the absence of substantial toxicity to said preadipocytes; and determining the presence or absence of an alteration in lipogenesis, lipolysis, and oxidation in the presence of said compound, wherein an alteration signifies said target.

13. The method of claim 11 wherein adipogenesis or differentiation of preadipocytes to adipocytes is determined by assaying for G3PD activity or PPAR γ activity.

14. The method of claim 11 wherein adipogenesis or differentiation of preadipocytes to adipocytes is determined by assaying for expression of a gene selected from the group consisting of PPARγ, C/EBPα, C/EBPβ, aP2, and GLUT4.

15. The method of claim 10 wherein lipogenesis is determined by measuring glucose to triglyceride conversion.

16. The method of claim 10 wherein lipogenesis is determined by measuring oleate to triglyceride conversion.

17. The method of claim 10 wherein lipolysis is determined by measuring release of labeled fatty acids from adipocytes.

18. The method of claim 10 wherein lipolysis is determined by measuring glycerol release from adipocytes.

19. The method of claim 10 wherein lipolysis is determined by measuring increased acrylodan-labeled intestinal fatty acid binding protein (ADIFAB) binding.

20. The method of claim 10 wherein oxidation is determined by measuring oxygen consumption.

21. A method for identifying a fat depot-specific inhibitor of human preadipocyte differentiation, comprising:
(a) identifying an inhibitor of human preadipocyte differentiation in a preadipocyte population derived from a first depot; and
(b) comparing the efficacy of said inhibitor in a preadipocyte population from a second depot, such that a fat depot-specific inhibitor of human preadipocyte differentiation is identified.

22. The method of claim 21, wherein the first depot is subcutaneous fat depot and the second depot is an omental or mesenteric fat depot.

23. A cell population comprising human preadipocytes, wherein said population is essentially pure.

24. The cell population of claim 23, wherein said population is at least 95% pure.

25. The cell population of claim 23, wherein said population is at least 98% pure.

26. The cell population of claim 23, wherein said population is at least 99% pure.

27. The cell population of claim 23, wherein the human preadipocytes are in suspension.

28. The cell population of claim 23, wherein the human preadipocytes are adhered to a cell culture surface.

29. The cell population of claim 23, wherein the human preadipocytes are of subcutaneous origin.

30. The cell population of claim 23, wherein the human preadipocytes are of mesenteric origin.

31. The cell population of claim 23, wherein the human preadipocytes are of omental origin.

32. A human preadipocyte cell strain which maintains differentiative capacity over at least 8 passages.

33. The cell strain of claim 32 which maintains differentiative capacity over at least 15 passages.

34. A human preadipocyte cell strain which maintains differentiative capacity over at least 25 passages.

35. A high-yield process for obtaining a human preadipocyte cell population which is essentially pure, comprising:
(a) isolating a mixed cell population from said fat tissue sample under conditions favoring a high preadipocyte yield; and
(b) removing contaminants from said mixed cell population, such that the essentially pure human preadipocyte cell population is obtained.

36. The process of claim 35, wherein the conditions favoring a high preadipocyte yield comprise:
(a) isolation within 0 to 24 hours following harvesting of said fat tissue; and
(b) removal of undigested connective tissue from the mixed cell population utilizing gauze filters.

37. The process of claim 35, wherein removing contaminants from said mixed cell population comprises:
(a) removing contaminating erythrocytes;
(b) removing contaminating adherent cells; and
(c) removing tissue and cellular debris.

38. The process of claim 37, wherein removing contaminating erythrocytes is accomplished by incubating the mixed cell population in an erythrocyte lysis buffer.

39. The process of claim 37, wherein removing contaminating adherent cells is accomplished by adhering the mixed cell population to a cell culture surface and preferentially trypsinizing the preadipocytes.

40. The process of claim 35, wherein said fat tissue is of subcutaneous origin.

41. The process of claim 35, wherein said fat tissue is of mesenteric origin.

42. The process of claim 40, wherein the conditions favoring a high preadipocyte yield further include coarsely mincing said fat tissue

43. The process of claim 35, wherein said fat tissue is of omental origin.

44. The process of claim 43, wherein the conditions favoring a high preadipocyte yield further include:
(a) finely mincing said fat tissue; and
(b) digesting said fat tissue in the presence of a semi-artificial serum supplement or bovine serum albumin.

45. The process of claim 35, wherein the essentially pure human preadipocyte cell population obtained is at least 85% pure.

46. The process of claim 35, wherein the essentially pure human preadipocyte cell population obtained is at least 90% pure.

47. The process of claim 35, wherein the essentially pure human preadipocyte cell population obtained is at least 95% pure.

48. The process of claim 35, wherein the yield is at least 10⁶ preadipocytes from 25 g of human fat tissue

49. The process of claim 35, wherein the yield is at least 10⁶ preadipocytes from 10 g of human fat tissue

50. The process of claim 35, wherein the yield is at least 10⁶ preadipocytes from 5 g of human fat tissue

51. The process of claim 35, wherein the yield is at least 10⁶ preadipocytes from 1 g of human fat tissue

52. A method of obtaining a highly-differentiated human adipocyte cell culture, comprising:
(a) obtaining a high-yield, essentially pure population of human preadipocytes;
(b) plating said preadipocytes at a density sufficient to ensure essentially 100% confluence upon adherence to the cell culture dish or plate;
(c) maintaining said preadipocytes in a serum-free differentiation medium such that a highly-differentiated human adipocyte cell culture is obtained.

53. The method of claim 52, wherein the cells are plated at a density of 3×10⁴ to 5×10⁴ cells per cm².

54. The method of claim 52, wherein the serum-free differentiation medium comprises a buffering component, glutamine, biotin, insulin, pantothenate, dexamethasone, triiodothyronine, rosiglitazone, fetuin, transferin, and isobutylmethylxanthine.

55. A method of obtaining a high-yield, essentially pure culture of human preadipocytes comprising;
(a) enzymatically dissociating a mixed-cell population from human fat tissue in serum-free, bovine serum (BSA)-containing isolation media;
(b) isolating the mixed-cell population from non-cell contaminants using gauze filters;
(c) treating the mixed-cell population with an erythrocyte lysis buffer prior to plating the cell population; and
(d) selectively trypsinizing the cell population and replating in a serum-free differentiation medium at a density sufficient to insure confluence.

56. A method of obtaining a highly-differentiated human adipocyte culture comprising obtaining a high-yield, essentially pure culture of human preadipocytes according to the method of claim 55 and further maintaining the culture in the serum-free differentiation medium such that a highly-differentiated human adipocyte cell culture is obtained.
